(19) United States
(12) Patent Application Publication
(10) Pub. No.: US 2026/0199690 A1
(43) Pub. Date: Jul. 16, 2026

(54) BRAIN IMPLANT DEVICES AND METHODS

(71) Applicants: Mayo Foundation for Medical Education and Research, Rochester, MN (US); Arizona Board of Regents on behalf of Arizona State University, Scottsdale, AZ (US)

(72) Inventors: Diogo P. Moniz Garcia, Jacksonville, FL (US); Alfredo Quinones-Hinojosa, Ponte Vedra Beach, FL (US); Jennifer Blain Christen, Chandler, AZ (US); Daniel Gulick, Tempe, AZ (US); Shiyi Liu, Tempe, AZ (US); Ahmed Abed Benbuk, Tempe, AZ (US)

(21) Appl. No.: 19/133,976

(22) PCT Filed: Dec. 1, 2023
(86) PCT No.: PCT/US2023/082117

§ 371 Date: May 29, 2025

Related U.S. Application Data
(60) Provisional application No. 63/429,539, filed on Dec. 1, 2022.

(51) Int. Cl. A61N 1/375; A61N 1/05; A61N 1/36; A61N 1/378

(52) U.S. Cl. CPC A61N 1/37514; A61N 1/0531; A61N 1/36002; A61N 1/3756; A61N 1/3787; A61N 1/36082

(57) ABSTRACT

A system for brain stimulation and monitoring, the system comprising: an implant comprising: an implant transceiver; a power management and storage unit; one or more electrodes; a recorder unit; and a memory; an external device comprising: a power source; and an external device transceiver configured to send power signals and stimulation signals to the implant transceiver; wherein the external device is configured to wirelessly send power signals and stimulation signals to the implant; wherein the implant is configured to record one or more biosignals from the one or more electrodes and wirelessly communicate the biosignals to the external device.

### CROSS-REFERENCE TO RELATED APPLICATIONS

This application claims the benefit of U.S. Provisional Application Ser. No. 63/429,539, filed Dec. 1, 2022. The disclosure of the prior application is considered part of the disclosure of this application, and is incorporated in its entirety into this application.

### BACKGROUND

### 1. Technical Field

This disclosure relates to systems, devices, and methods for brain implants. For example, this disclosure relates to brain implants that facilitate stimulation, monitoring, and treatment of a subject.

### 2. Background Information

Tumor-related seizures affect a proportion of tumor patients with prevalence ranging from around 90% in low-grade gliomas, to around 50-60% in high-grade gliomas. Therapeutic approaches can be limited with a proportion of patients remaining treatment resistant. Early diagnosis and monitoring can help prevent progression into refractory epilepsy. Some seizure alert devices utilize wearables that detect limb movement. These seizure alert devices are unable to detect subclinical seizures, thus preventing pre-convulsion therapeutic intervention.

Motor rehabilitation in patients with acquired deficits after stroke, trauma, or tumor remains limited in a large subset of patients, with a lack of effective therapeutic modalities available. Stroke constitutes a major public health problem affecting millions worldwide, with up to 40% presenting with chronic motor deficits that are refractory to current rehabilitation programs. Motor deficits are also common in trauma and tumor patients. Tumor patients acquire particular importance, as despite current pre-operative and intra-operative localization of motor eloquence, iatrogenic motor deficits continue to be observed in lesions affecting perirolandic regions and descending motor tracts and have been associated with worse survival in high-grade gliomas. However, current management of these deficits is hindered by the lack of clearly effective therapeutic adjuncts beyond standard outpatient and inpatient rehabilitation programs.

Approaches to enhance motor rehabilitation include robotic-assisted rehabilitation, muscular electrical stimulation, brain stimulation, and brain-computer interface. Brain stimulation enables adaptive brain plasticity during rehabilitation training, modifying local cortical excitability, promoting focal and remote neuroplasticity, and correcting maladaptive changes. Increased motor recovery in preclinical animal models with brain stimulation has also shown structural changes, including increase in synaptic density and increased synaptic response in perilesional cortex. Noninvasive brain stimulation methods such as transcranial magnetic stimulation (TMS) and transcranial direct current stimulation (tDCS) (including cathodal and anodal), have been studied with functional improvement being limited to 10-30% and being short-lasting. The extent or spread of the induced current in the brain may be variable and difficult to assess without in vivo stimulation and recording studies, which in turn prevents adequate determination of which cortical neurons and which cortical areas are affected with each TMS pulse. Furthermore, this approach can be limited to superficial brain areas that can be targeted, with limited effect on subcortical regions. Invasive implants can overcome some of the limitations observed with non-invasive stimulation approaches, by attaining high temporal and spatial resolution, sufficient intensity, and continuous stimulation throughout task-oriented long-term motor training for home-based use. However, human clinical studies mainly conducted in stroke patients have been inconsistent, at least partly due to location-dependent differences. While most preclinical studies have been conducted in stroke animal models with restricted cortical impairment, human studies have not consistently stratified patients based on the location of impairment, with enrolled patients including damage to subcortical areas and even descending motor tracts, with associated dysfunction to widespread cortical areas outside the initial infarct. Current implants are also hindered by the high prevalence of side effects observed, including lead failure, migration, and infection.

### SUMMARY

This disclosure describes systems, devices, and methods for brain stimulation, treatments, and monitoring.

Some embodiments described herein include a system for brain stimulation and monitoring. The system can include an implant that includes: an implant transceiver, a power management and storage unit, one or more electrodes, a recorder unit, and a memory. The system also includes an external device that can include: a power source, and an external device transceiver configured to send power signals and stimulation signals to the implant transceiver. The external device is configured to wirelessly send power signals and stimulation signals to the implant; where the implant is configured to record one or more biosignals from one or more electrodes and wirelessly communicate the biosignals to the external device.

Such a system can optionally include one or more of the following features. The system where the implant is battery-less. The implant can include a flexible substrate that connects to one or more electrodes, the implant transceiver, and the power management and storage unit. The flexible substrate can include a biocompatible material. The implant is configured to be positioned between a bone layer and the cortex of the skull of a subject.

Some embodiments described herein include a method of stimulating and monitoring a brain of a subject. The method includes positioning an implant between a bone layer and a cortex of the skull of a subject; communicating wireless power signals from an external device to the implant, converting the wireless power signals into stored power at the implant, communicating wireless stimulation signals from the external device to the implant, processing the stimulation signals at the implant into stimulation voltages delivered by one or more electrodes at the implant, recording one or more biosignals at the one or more electrodes, storing the one or more biosignals at the implant, and communicating the one or more biosignals wirelessly from the implant to the external device.

Such a system can optionally include one or more of the following features. The method where the implant is battery-less. The implant can include a flexible substrate that connects to one or more electrodes, a transceiver, and a power management and storage unit. The flexible substrate can include a biocompatible material.

Some embodiments described herein include a device for brain stimulation and monitoring an implant configured to be positioned between a bone layer and a cortex of a skull of a subject, the implant can include: a flexible substrate that can include a biocompatible material; an implant transceiver connected to the flexible substrate; a power management and storage unit connected to the flexible substrate; one or more electrodes connected to the flexible substrate, the one or more electrodes are configured to deliver stimulation voltages to the cortex of the subject and to record biosignals from the cortex of the subject; a recorder unit connected to the flexible substrate; and a memory connected to the flexible substrate. The device also includes where the implant is configured to wirelessly communicate with an external device.

Some embodiments described herein include a device for brain treatment an implant configured to be positioned between a bone layer and a cortex of a skull of a subject, the implant can include: a flexible substrate that can include a biocompatible material; an implant transceiver connected to the flexible substrate; a power management and storage unit connected to the flexible substrate; one or more electrodes connected to the flexible substrate, the one or more electrodes are configured to create an electrical field that delivers stimulation voltages to a treatment area in the cortex of the subject; a recorder unit connected to the flexible substrate; and a memory connected to the flexible substrate. The device also includes where the implant is configured to wirelessly communicate with an external device.

Such a system can optionally include one or more of the following features. The device where the implant is battery-less. The implant can include a flexible substrate that connects to one or more electrodes, the implant transceiver, and the power management and storage unit. The flexible substrate can include a biocompatible material. The implant is configured to be positioned between a bone layer and the cortex of the skull of a subject.

Some embodiments described herein include a system for brain treatment an implant can include: an implant transceiver, a power management and storage unit, one or more electrodes, a recorder unit, and a memory. The system also includes an external device can include: a power source, and an external device transceiver configured to send power signals and stimulation signals to the implant transceiver. The system also includes where, responsive to the power signals and the stimulation signals, the implant generates an electrical field from one or more electrodes in a treatment area.

Such a system can optionally include one or more of the following features. The system where the treatment area is a tumor. The implant can include a plurality of electrode pairs. The treatment area includes a plurality of tumors.

Particular embodiments of the subject matter described in this document can be implemented to realize one or more of the following advantages. The disclosed systems including the implantable devices can be positioned at the time of surgery and can be used continuously. The implant can be positioned between the bone of the skull and the dura. The implant can facilitate a reduced risk of infection, migration and other adverse events. The implant can have direct access to the cortical surface, separated by a dura graft. The implant can have increased temporal and spatial sensitivity, allowing for high-precision stimulation or monitoring. Epidural placement of the implant facilitates an improved patient experience by removing the implementation of an external helmet or large power source.

Some embodiments described herein advantageously provide a “battery-less” implant. As used herein “battery-less” means that the external device both powers and sends stimulation protocols and signals to the implant without a power cable extending from the external device to the implant. The implant receives, manages, and outputs the wirelessly received power signals or signals derived from the received power signals. Additionally, the implant wirelessly transmits the image data acquired by the implant to the external device without a data cable extending from the implant. As such, the transmission of the power, stimulation protocols, and data between the implant and the external device are achieved without wires or power cords extending between the external device and the implant.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting. The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1A is a side view of an example stimulation and monitoring system in accordance with some embodiments.

FIG. 1B is a sectional view of the stimulation and monitoring system of FIG. 1A.

FIG. 1C is a side view of an example stimulation and monitoring system in accordance with some embodiments.

FIG. 1D is a side view of an example stimulation and monitoring system in accordance with some embodiments.

FIG. 2A is a schematic diagram of an example stimulation and monitoring system.

FIG. 2B is a schematic diagram of an example stimulation and monitoring system with a single-channel operation.

FIG. 2C is a schematic diagram of an example stimulation and monitoring system with a multi-channel operation.

FIG. 2D is a circuit diagram of the stimulation and monitoring system of FIG. 2A.

FIG. 3 is a perspective view of an example implant device, in accordance with some embodiments.

FIG. 4A is a perspective view of an example implant device, in accordance with some embodiments.

FIG. 4B is a perspective view of an example implant device, in accordance with some embodiments.

FIG. 4C is a side view of a cross section of an example implant device, in accordance with some embodiments.

FIGS. 4D-F are perspective views of the implant device of FIG. 4B.

FIG. 5A is exemplary testing results of an example stimulation and monitoring system.

FIG. 5B is an exemplary stimulation protocol for motor response, in accordance with some embodiments.

FIG. 5C is an exemplary individual pulse train, in accordance with some embodiments.

FIG. 6A is exemplary testing results of an example stimulation and monitoring system.

FIG. 6B is exemplary testing results of an example stimulation and monitoring system.

FIG. 6C is exemplary testing results of an example stimulation and monitoring system.

FIG. 6D is exemplary testing results of an example stimulation and monitoring system.

FIG. 6E is exemplary testing results of an example stimulation and monitoring system.

FIG. 6F is exemplary testing results of an example stimulation and monitoring system.

FIG. 7 is a perspective view of an example implant device, in accordance with some embodiments.

FIG. 8A is exemplary fabrication steps of an example implant device, in accordance with some embodiments.

FIG. 8B is exemplary fabrication steps of the example implant device of FIG. 8A, in accordance with some embodiments.

FIG. 8C is an exemplary charge balancing circuit and exemplary results, in accordance with some embodiments.

FIG. 9A is a schematic diagram of an example stimulation and monitoring system.

FIG. 9B is exemplary results of an off-center-fed antenna, in accordance with some embodiments.

FIG. 9C is a circuit diagram of the stimulation and monitoring system of FIG. 9A.

FIG. 10 is a perspective view of an example external transceiver system, in accordance with some embodiments.

FIG. 11A is exemplary testing results of an example stimulation and monitoring system.

FIG. 11B is exemplary testing results of an example stimulation and monitoring system.

FIG. 11C is exemplary testing results of an example stimulation and monitoring system.

FIG. 12A is a side view of an example stimulation and monitoring system in accordance with some embodiments.

FIG. 12B is a perspective view of an example stimulation and monitoring implant, in accordance with some embodiments.

FIG. 12C is a perspective view of an example stimulation and monitoring system in accordance with some embodiments.

FIG. 13A is a side view of an example stimulation and monitoring system in accordance with some embodiments.

FIG. 13B is a perspective view of an example stimulation and monitoring implant, in accordance with some embodiments.

FIG. 14A is a schematic diagram of an example stimulation and monitoring system.

FIG. 14B is a circuit diagram of the stimulation and monitoring system of FIG. 2A.

FIG. 14C is a schematic diagram of an example stimulation and monitoring system.

FIG. 14D is a schematic diagram of an example stimulation and monitoring system.

Like reference numbers represent corresponding parts throughout.

### DETAILED DESCRIPTION

This disclosure describes systems, devices and methods for brain implants. For example, this disclosure relates to brain implants that facilitate monitoring, stimulation, and treatment of a subject.

Embodiments of this disclosure include a system for brain stimulation and monitoring that includes an implant and an external device. The implant includes an implant transceiver, a power management and storage unit, one or more electrodes, a recorder unit, and a memory. The external device includes an external device transceiver and a power source. The external device and the implant are configured to wirelessly communicate with each other between the implant transceiver and the external device transceiver. The devices, implants, and systems described herein can be implemented in the monitoring, stimulation, and treatment of subjects having epilepsy, tumors, neurodegenerative disorders, neuroinflammatory diseases or disorders, and trauma. The devices, implants, and systems described herein can be implemented for various cortical and subcortical functions such as speech, motor, sensory, vision, and others.

In some embodiments, the systems described herein facilitate the placement of the implant devices at the time of surgery. The implant devices can be placed on a dural substitute, and the dural substitute with the implant device can be secured in position during surgery with a reduced or eliminated risk of migration or malpositioning of the implant devices post-surgery. The dural substitute with the implant device can be positioned between the skull (i.e., bone) and the dura of the subject, the positioning between the skull (i.e., bone) and the dura can remove and/or reduce the risk for infection and other local complications. In some aspects, the dural substitute can be a dural graft or artificial dura.

In some embodiments, the implant device has direct access to the brain surface (i.e., can be positioned underneath the bone surface and have access to the cortex), and can be separated from the cortex by a dural substitute. The implant device can facilitate long-term invasive monitoring (e.g., with applications in traumatic brain injury, stroke, tumor and epilepsy), stimulation to aid in motor rehabilitation after stroke, brain traumatic injury, or iatrogenic deficits detected during surgery. By including modifications to our implant including an associated electrode placed beyond the bone flap and connected to a subcutaneous battery placed as per standard in deep brain stimulation surgery, our invention will allow for continuous monitoring and communication with the patient's physician creating an opportunity for an individualized center for digital health.

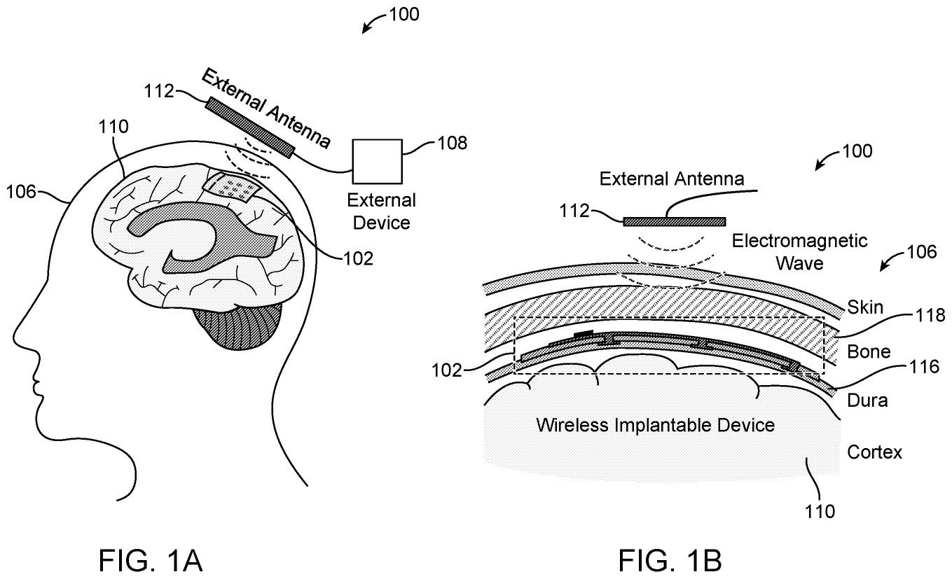

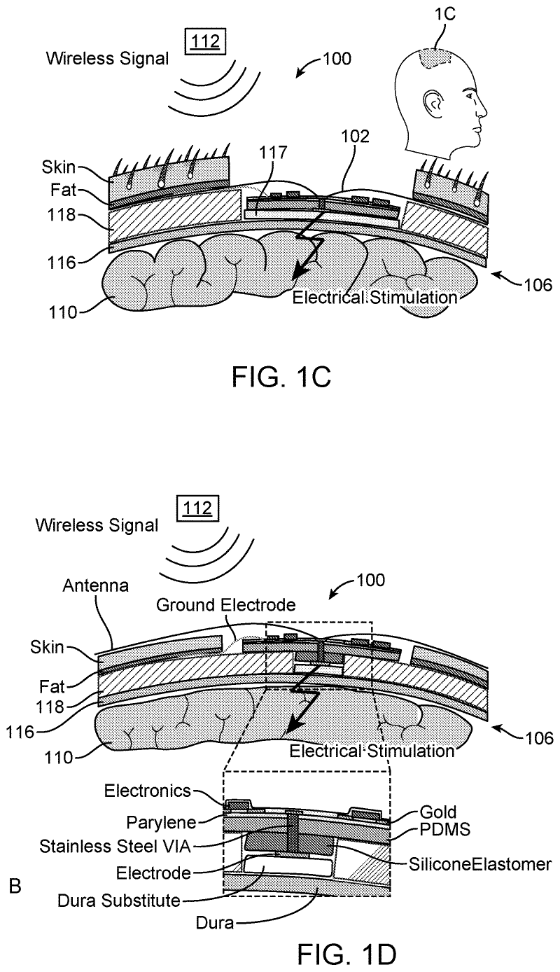

Referring to FIGS. 1A and 1B, a system 100 for brain stimulation and monitoring is shown. The system 100 includes an implant 102 and an external device 108. The implant 102 is positioned in a head 106 of a subject underneath the bone surface 118, and has access to the cortex 110 of the subject. The implant 102 can be a wireless implant that includes a thin, flexible, and biocompatible material, and the implant 102 is operable to record neural activity (e.g., biosignals) and to deliver stimulation to the brain of the subject. The external device 108 includes an external device transceiver 112 that facilitates wireless communication with the implant 102 (e.g., through electromagnetic or inductive coupling). In some aspects, the external device 108 can be a wearable device that sits behind an ear of a subject.

The positioning of the implant 102 within the layers of the head 106 of the subject is illustrated. In some aspects, the implant 102 can be directly positioned on the dura mater 116 or dural substitutes. The implant 102 has a low profile and high flexibility that facilitate the ability of the implant 102 to contour to the curved surface of the dura 116, ensuring a stable contact between one or more electrodes of the implant 102 and the tissue of the dura 116. In some aspects, the implant 102 can also be integrated onto an artificial dura graft, which can be used as a substitute for repairing the dura mater. In some aspects, the implant 102 can be on top of a dura graft, underneath a dura graft, or sandwiched between two layers of a dura graft. The implant 102 can be implanted under the skull 118 for long-term neural recording and stimulating operations without intracranial wiring. In some aspects, the absence of intracranial wiring reduces the risk of infections.

The placement of the implant 102 facilitates several advantages, including continuous use, reduction of migration, reduction of infection, reduction of other adverse events, increased spatial and temporal sensitivity. For example, the implant 102 can be used continuously. The placement of the implant 102 between the bone and the dura reduces the risk of infection, the risk of migration, and the risk of other adverse events. The implant 102 can have increased temporal and spatial sensitivity that is facilitated by the direct access to the cortical surface. The increased temporal and spatial sensitivity facilitates high precision stimulation or monitoring by the implant 102.

The implant 102 can be positioned on a dural substitute and can facilitate direct cortical and subcortical stimulation to aid in rehabilitation, for extended monitoring, and/or for life-long term monitoring. By placing it epidurally within a dural substitute, the implant 102 provides high sensitivity and accuracy of direct stimulation and monitoring with improved clinical outcomes.

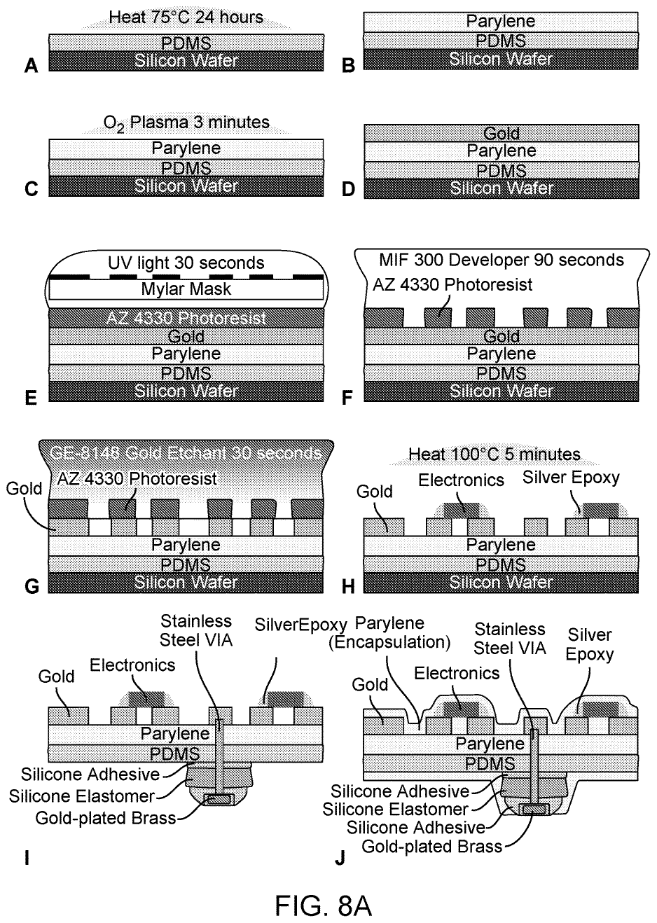

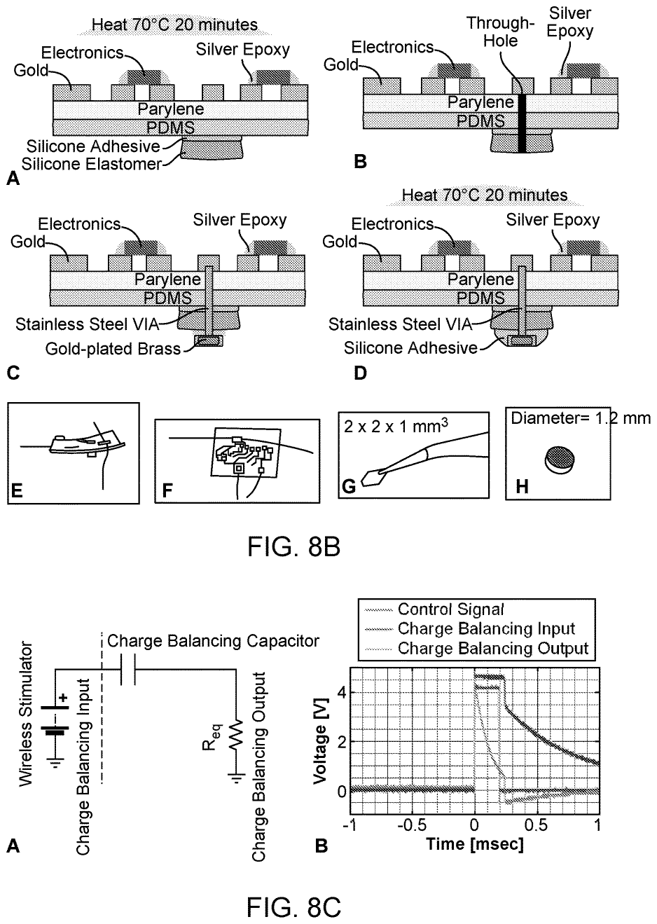

Referring to FIGS. 1C and 1D, the positioning of the implant 102 within the layers of the head 106 of the subject is illustrated. In some aspects, the implant 102 can be directly positioned on the dura mater 116 or dural substitutes 117 (FIG. 1C). The implant 102 has a low profile and high flexibility that facilitate the ability of the implant 102 to contour to the curved surface of the dura 116, ensuring a stable contact between one or more electrodes of the implant 102 and the tissue of the dura 116. In some aspects, the implant 102 can be on top of a dura graft, underneath a dura graft, or sandwiched between two layers of a dura graft. The implant 102 can be implanted under the skull 118 for long-term neural recording and stimulating operations without intracranial wiring. In some aspects, the absence of intracranial wiring reduces the risk of infections. The implant 102 can be implanted on the bone 118 to facilitate ease of access and operation. The implant 102 can include several layers as described in more detail below (e.g., see FIGS. 8A and 8B). For example, the implant 102 includes electronics, gold, parylene, Polydimethylsiloxane (PDMS), stainless steel VIA, silicone elastomer, and one or more electrodes.

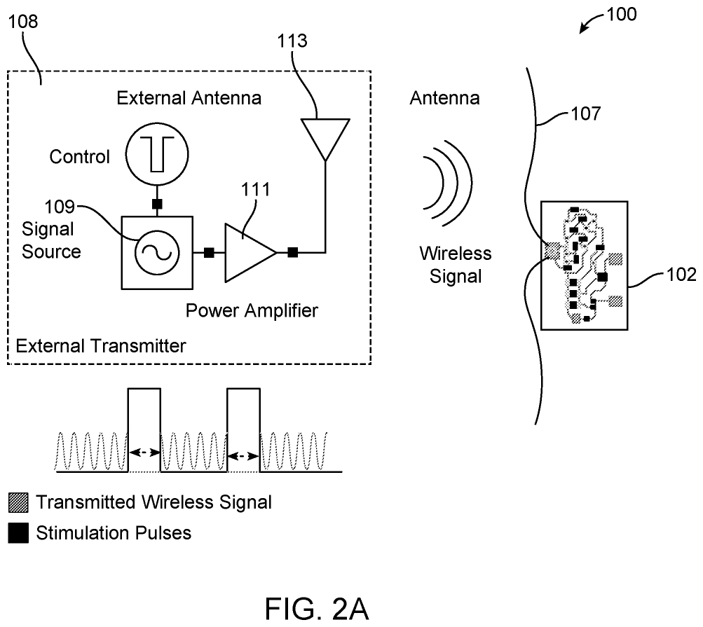

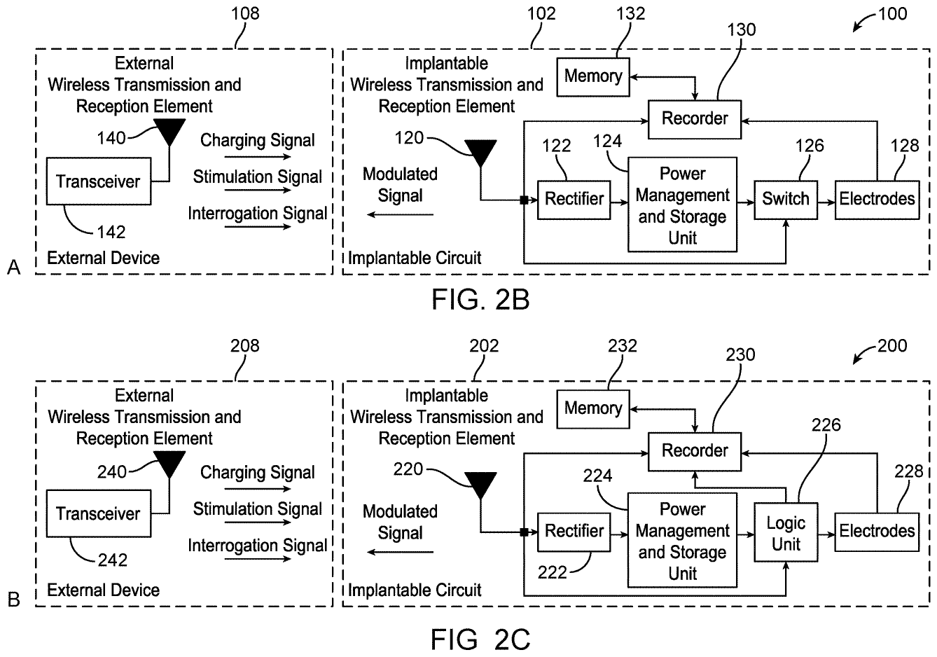

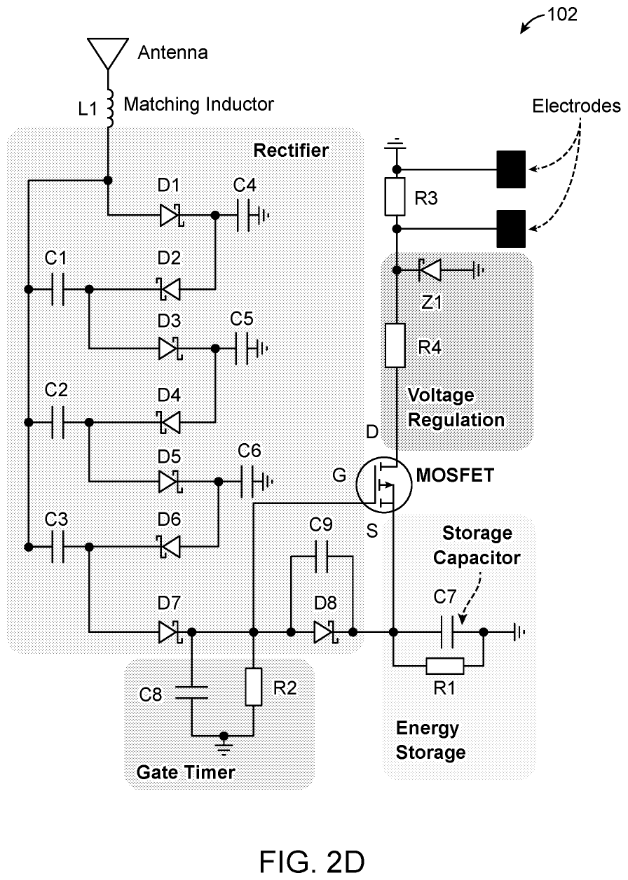

Referring to FIG. 2A, exemplary system 100 can include the implant 102 and external device 108. The implant 102 can receive wireless power through a dipole antenna 107. The implant 102 incudes a rectifier, energy storage capacitors, and a P-MOSFET to generate stimulation pulses.

The external device 108 delivers power to the implant 102 and controls the parameters of the monophasic voltage stimulation pulses. These parameters include amplitude, frequency, and pulse width. The external device 108 includes an RF signal source 109 (e.g., 5009, Valon) that generates a continuous wave radio frequency (RF) signal at or around 2.45 GHz. A power amplifier 111 (e.g., MPA-24-20, RF Bay, Inc) boosts the RF power level that is radiated by an external antenna 113 (e.g., A10194, Antenova). An On-Off-Keying (OOK) signal can control the wireless transmission. During the ON periods, the external device 108 delivers wireless power to the implant 102 to replenish the implant's energy storage. The implant 102 generates monophasic stimulation pulses during the OOK OFF period, delivering the stored energy through the electrodes. The stimulation pulse width is approximately equal to the OOK OFF period. The implant 102 remains idle if the OOK transmission ceases. This approach facilitates on-demand operation and the ability to change stimulation parameters any time after the implant 102 is placed on the dura substitute.

Referring to FIGS. 2B and 2C, example schematic diagrams of exemplary systems 100 and 200 are shown. Each system 100, 200 can be implanted into the head 106 of the subject as described in reference to FIGS. 1A and 1B. The system 100 includes a single-channel configuration (FIG. 2B) and the system 200 includes a multichannel configuration (FIG. 2C).

The implants 102, 202 can include components that facilitate harvesting energy, channel selection, stimulation, and recording. The implants 102, 202 may include any or all these units, depending on whether the implants 102, 202 are for stimulation only, recording only, or combined stimulation and recording.

The implants 102, 202 can include a wireless transmission and reception element 120, 220 that converts a wireless signal between the wireless and wired domain. For example, the wireless transmission/reception element 120, 220 may be implemented as an antenna to capture or radiate a wireless signal in the radio frequency spectrum, or it can be implemented as a coil to capture or radiate a wireless signal through inductive coupling.

The implants 102, 202 can include a rectifier 122, 222 that converts the signal that is captured using the wireless reception element 120, 220 into DC voltage to be used to deliver electrical stimulation or to power the circuit components that require an energy source to operate. For example, the rectifier 122, 222 may be implemented in a Dickson or Greinacher voltage multiplier or a diode bridge configuration.

The implants 102, 202 can include a power management and storage unit 124, 224 that is used to store the DC voltage. The power management and storage unit 124, 224 may include a storage capacitor, boost converter, buck converter, buck-boost converter, a voltage regulator, or other forms of power managing circuit to manage the DC voltage.

The implant 102 can include a switch 126 that can control the delivery of the stored energy to one or more electrodes 128 when the implant 102 is a single-channel stimulator or recorder (FIG. 2B). Responsive to the switch 126 positioned in the OFF state, the energy is kept in the power management and storage unit 124. The energy is delivered to one or more electrodes 128 when the switch is in the ON position.

The implant 202 can include a logic unit 226 can select and control the delivery of the stored energy to a specific pair of electrodes 228 out of several pairs of electrodes 228, and to select a specific pair of electrodes 228 to record biosignals. The logic unit 226 is implemented if the implant 202 is used for multi-channel stimulation and/or recording (FIG. 2C). For example, the logic unit 226 may include processing and memory elements such as a microcontroller, shift register, multiplexer, or bit-correlator.

The implants 102, 202 can include one pair of electrodes in single-channel operation (FIG. 2B) that is used to deliver stimulation to and acquire biosignals from the target. Several electrode pairs are implemented in multi-channel operation (FIG. 2C).

The implants 102, 202 can include a recorder unit 130, 230 that can modulate a carrier signal with the biosignals it acquires from an electrode pair (e.g., electrodes 128, 228). The recorder unit 130, 230 can be implemented using a passive component or an active component that is powered through the stored energy. For example, a passive recorder can be implemented in a non-linear element to generate mixing harmonics. An active recorder can be an active modulator, such as a frequency modulator. The modulated signal that is generated by the recorder 130, 230 can be transmitted via the wireless transmission and reception element 120, 220.

The implants 102, 202 can include a memory unit 132, 232 that may be integrated with the recorder 130, 230 to store the biosignals acquired from the electrodes 128, 228. For example, the memory unit 132, 232 may include an analog-to-digital converter (ADC) to convert analog biosignals and store them in a memory bank as bits.

The external devices 108, 208 can wirelessly communicate a variety of signals with the implant 102, 202. For example, the external device 108, 208 generates and transmits a charging signal, an interrogation signal, and a stimulation signal to the implant 102, 202.

The external devices 108, 208 can include an external wireless transmission and reception element 140, 240 that converts a wireless signal between the wireless and wired domain. For example, the wireless transmission/reception element 140, 240 can be implemented in an antenna to capture and radiate a wireless signal in the radio frequency spectrum, or it can be implemented in a coil to capture and radiate a wireless signal through inductive coupling. In some aspects, the external wireless transmission and reception element 140, 240 can communicate with the implants 102, 202 via the implant wireless transmission and reception element 120, 220. In some examples. the wireless communication between the external device 108, 208 and the implant 102, 202 can occur with a distance from 10 cm to 30 cm between the external device 108, 208 and the implant 102, 202.

The external devices 108, 208 can include a transceiver 142, 242 that generates the charging signal, the interrogation signal, and the stimulation signal, and receives the modulated recording signal from the implant 102, 202 that carries the acquired biosignals from the electrodes 128, 228.

In some aspects, the external devices 108, 208 perform operations that direct the implants 102, 202 to perform operations. For example, the external devices 108, 208 perform a method that includes charging, stimulation, and interrogation of the respective implants 102, 202 using an external transceiver (e.g., transceivers 142, 242). In some aspects, the external device 108, 208 delivers energy to the implant 102, 202 via a charging signal to charge the power management and storage unit 124, 224.

In some aspects, the external device 108 delivers a stimulation signal that activates the switch 126 from the OFF state to the ON state to deliver stimulation to a pair of electrodes 128 for single-channel operation in implant 102. In multi-channel operation of implant 202, the external device 208 delivers a stimulation signal that instructs the logic unit 226 to select a specific pair of electrodes out of several pairs of electrodes 228 to deliver the stimulation.

In some aspects, the external device 108, 208 delivers an interrogation signal that instructs the recorder 130, 230 to modulate a carrier signal with the biosignals that are acquired from an electrode pair 128, 228. In multi-channel operation of implant 202, the interrogation signal instructs the logic unit 226 to activate recording from a specific pair of electrodes out of several pairs of electrodes 228. The recorder 230 may generate the carrier signal, and modulate the interrogation signal with the biosignals that are acquired from the electrodes 228. The interrogation signal instructs the recorder 230 to modulate a carrier signal with the data that is stored in the memory 232

Referring to FIG. 2D, a circuit diagram of the implant 102 is shown. A Dickson voltage multiplier rectifies and boosts the received wireless signal. It includes several Schottky diodes (D1-D8, e.g., JDH2S02SL, Toshiba) and smoothing capacitors (C1-6, e.g., 10 pF, Murata). The number of stages facilitates a balance between high output voltage and a small footprint. An impedance matching inductor (L1, e.g., 2.2 nH) cancels the negative imaginary impedance at the input of the Dickson multiplier to minimize the input reflection coefficient seen by the antenna looking into the rectifier. An initial value for the optimal matching inductor can be 3 nH (e.g., Advanced Design System 2021, Keysight).

A timer circuit (C8, R2) can be applied to the gate of the P-MOSFET (e.g., PMZ320UPEYL, Nexperia). The timer circuit can charge during the OOK ON intervals and to a voltage that matches the voltage formed across the energy storage capacitor (C7, e.g., 30 μF, Murata). Therefore, VDS~0 and no current is delivered to the load resistor (R3, 10 kΩ). At the onset of the OOK OFF intervals, the timer circuit discharges, and current is delivered through the drain-source channel to the load. A 2.45 GHz signal can be coupled directly to the traces from the external antenna. An RF short capacitor (C9, 20 pF) is connected in parallel across diode D8 to short the coupled 2.45 GHz signal and ensure that the gate is biased using the gate timer circuit. In addition, this capacitor is a DC block that keeps the DC voltage at C7 from appearing at the MOSFET gate.

An optional voltage regulation circuit can be implemented at the output to limit the amplitude of the stimulation pulses and ensure safety. The voltage regulation circuit can include a Zener diode (Z1) with a specific voltage (Vz) that is determined by the medical application, and a tuning resistor (R4) to determine the regulated input voltage range. Neurostimulation intensity can be adjusted to achieve the desired effect based on the location of the electrode relative to the targeted neural structures. The Zener diode regulation scheme includes a fixed voltage. To adjust the stimulation for each subject's specific motor threshold, the pulse duration can be changed.

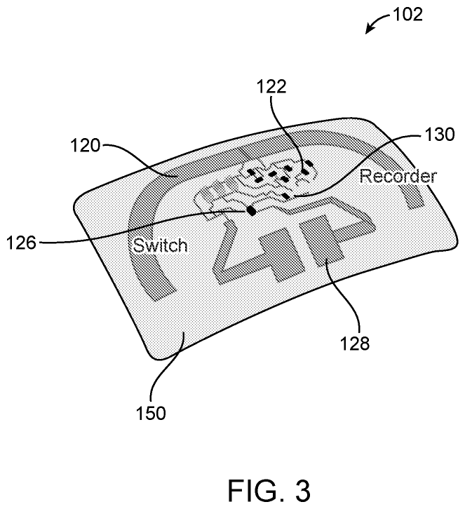

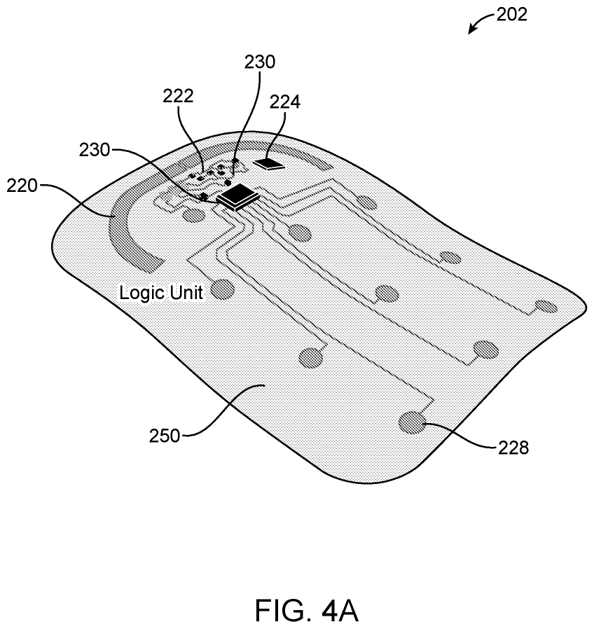

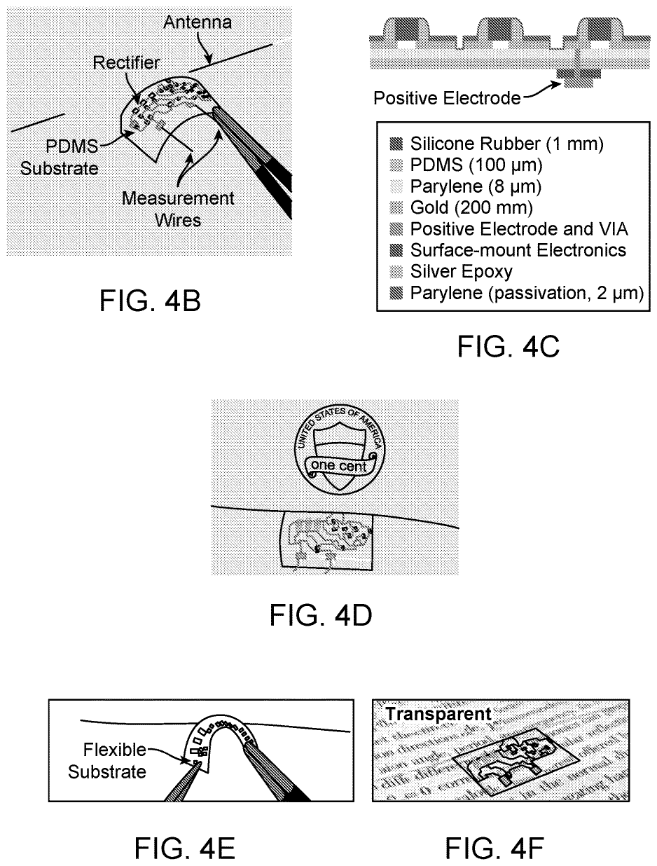

Referring to FIG. 3, a perspective view of the implant 102 is shown. Referring to FIG. 4A, a perspective view of the implant 202 is shown. In both embodiments shown in FIGS. 3 and 4A (e.g., the implants 102, 202), the wireless transmission and reception element 120, 220 can be a printed antenna. In some aspects, the printed antenna can be a planar dipole antenna, a folded dipole antenna, a loop antenna, a monopole antenna, a wire antenna, or patch antenna, or other forms of antenna. In some aspects, the wireless transmission and reception element 120, 220 can be an inductive coil. The implants 102, 202 each include a flexible substrate 150, 250 that can be made from highly flexible and stretchable materials such as silicone rubber, Ecoflex, or Polydimethylsiloxane (PDMS). The flexible substrate 150, 250 can also be made from flexible polymers such as Polyimide or Parylene C. The electrodes 128, 228 can include biocompatible metals such as gold, titanium, and platinum. The shape of the electrodes 128, 228 can be circle, rectangle, ring, or other electrode shape.

Referring to FIGS. 4B-G, the implant 102 can include a transparent, flexible, and biocompatible substrate (e.g., Parylene/PDMS).

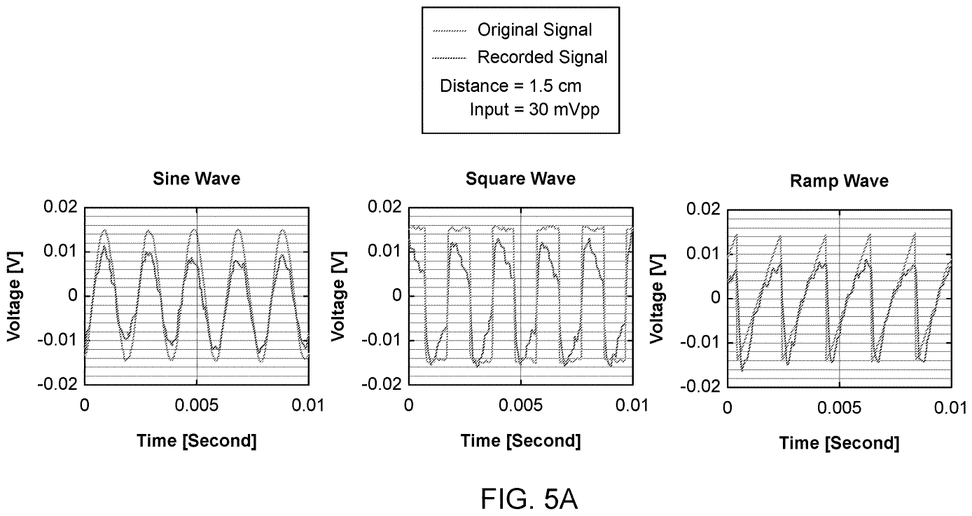

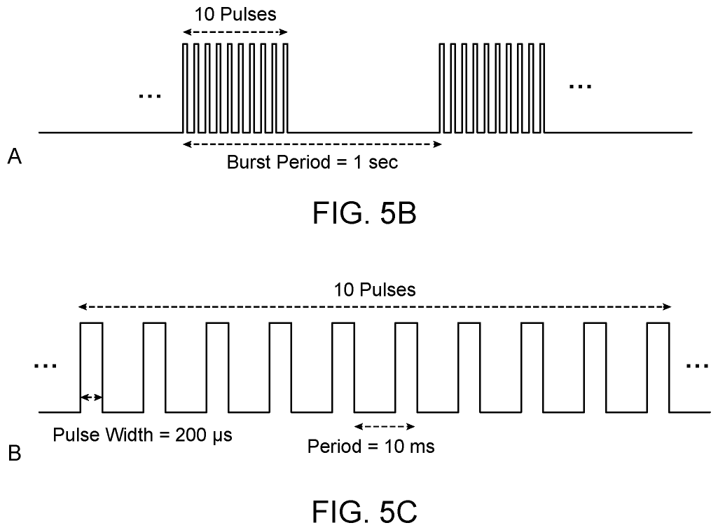

Referring to FIG. 5A, exemplary testing results are illustrated for the systems described herein. An arbitrary function generator generated emulated neural signals in three different waveforms including sine, square, and ramp, which were input to the sensing electrode (e.g. electrodes 128) of the implant 102. The external antenna 112 was positioned 1.5 cm away from the implant 102. As illustrated in FIG. 5, the wireless output signal aligned with the input under each type of waveform, demonstrating the feasibility of wireless battery-free neural recording.

Referring to FIGS. 5B-C, an example wireless stimulation protocol can be implemented to trigger a motor response in the subject. For example, FIG. 5B shows an example 10-pulse train transmitted at 1 Hz. FIG. 5C shows example parameters of the individual pulse train of FIG. 5B. In some embodiments, the signal was OOK-modulated using a function generator (33250A, Agilent) to obtain a burst of ten RF pulses each with a width of 200 μs at a frequency of 100 Hz. The RF burst was transmitted at a frequency of 1 Hz. Varying the pulse protocol affects the motor response threshold, with a wide parameter space for potential exploration. The transmit power can be adjusted until a motor response was triggered. The external antenna with dimensions of 10×10 mm² and gain of 4.1 dBi was placed at a distance of 20 mm away from the wireless implant.

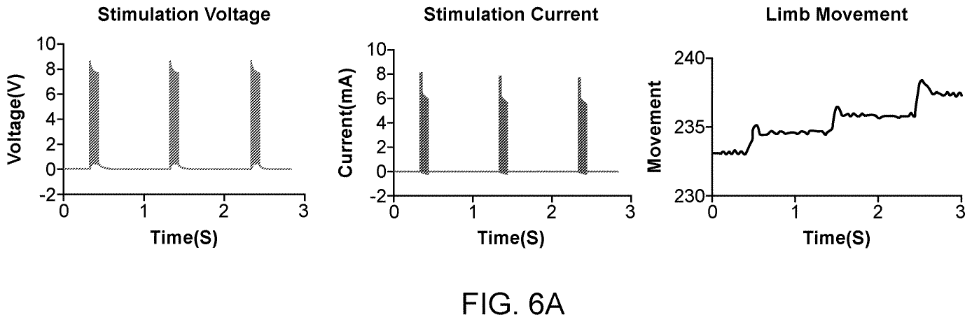

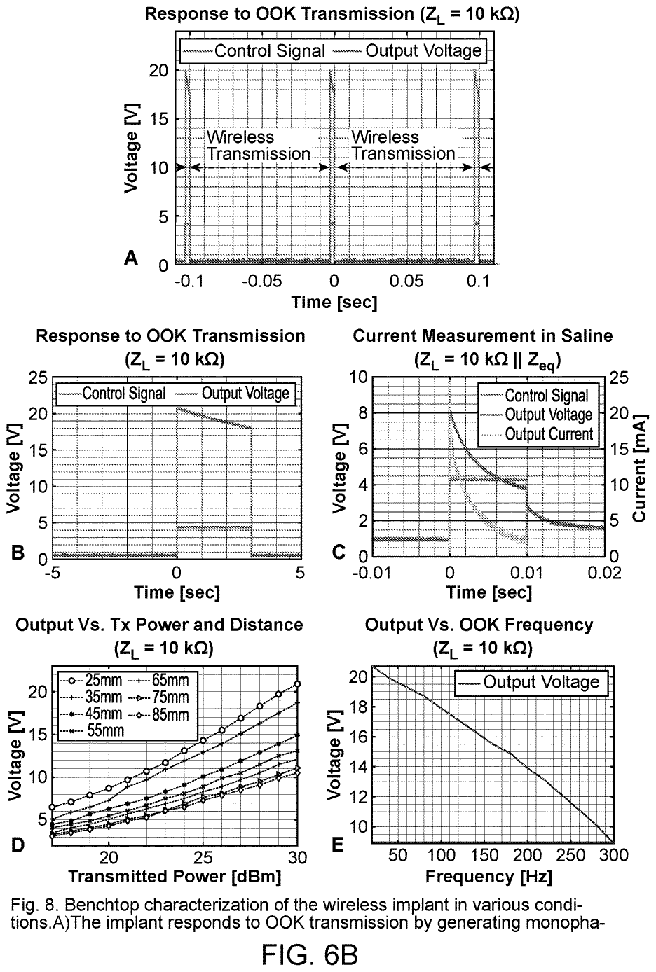

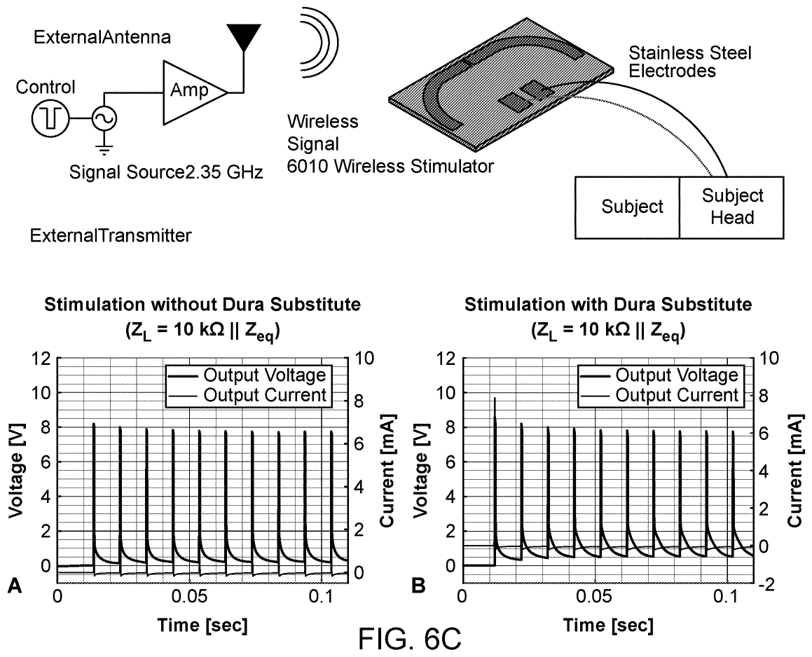

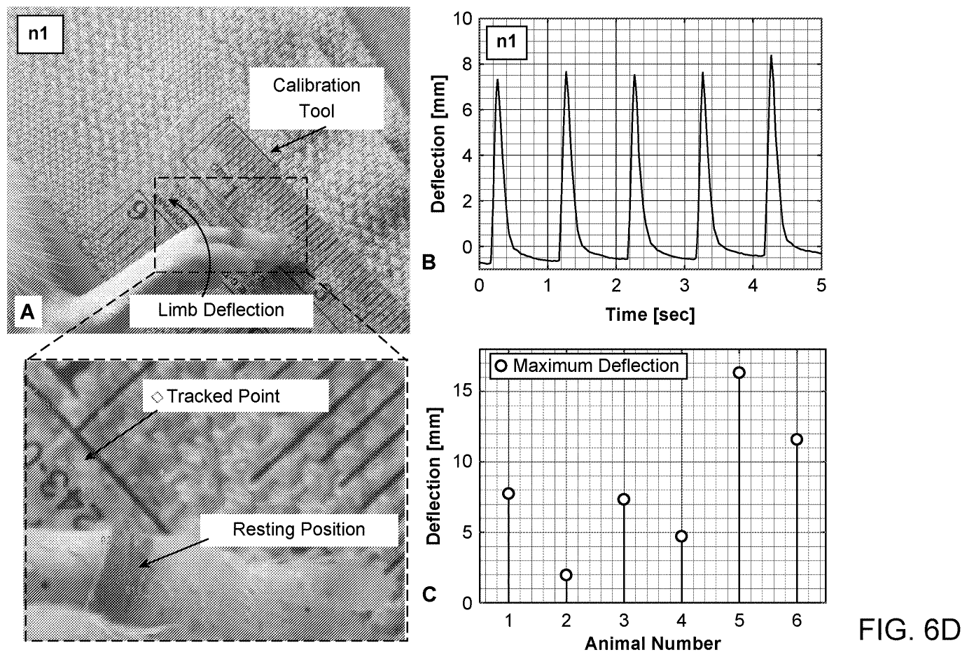

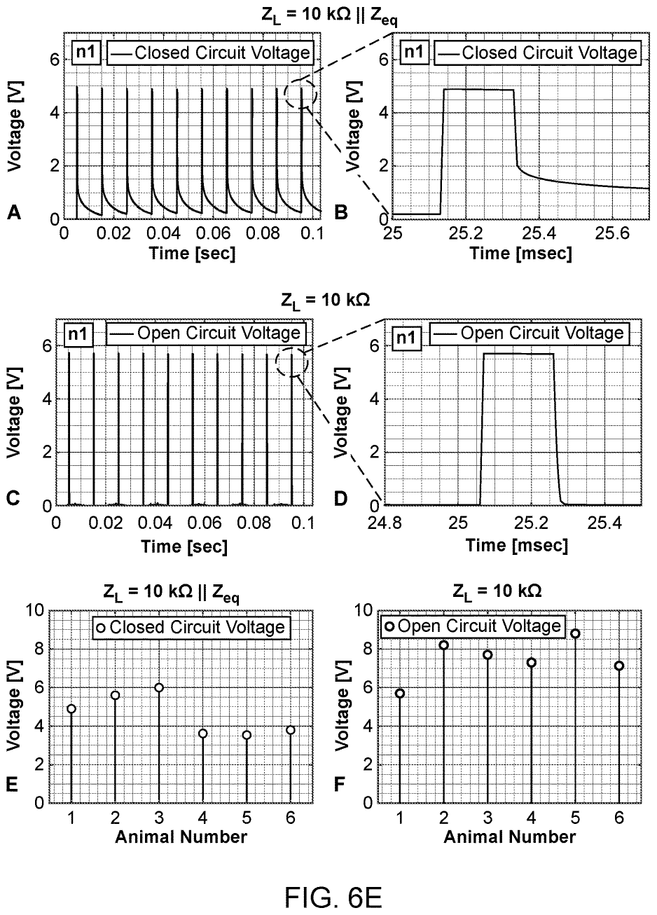

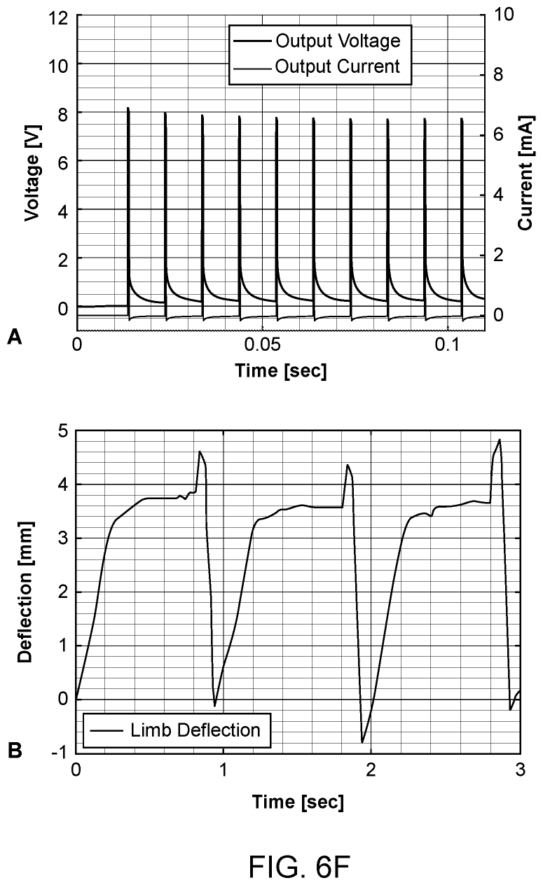

Referring to FIG. 6A, results of stimulation testing are shown. The wireless stimulation function of the implant (e.g., implant 102, 202) was verified in-vivo on a rat model. A cranial window was opened over the right motor cortex region of the rat and an artificial dura graft patch was used to close the surgical wound. Two stainless steel wire electrodes were placed onto the dura graft to deliver electrical stimulation signals generated from the implant (e.g., implant 102, 202).

FIG. 6A shows the voltage/current stimulus generated by the implant and the recorded movement from the Rat hindlimb. The limb movement demonstrated well-controlled synchronization with the stimulation signals, proving the implant can deliver sufficient electrical stimulus to the motor cortex through the artificial dura graft.

Referring to FIG. 6B, results of stimulation testing are shown. The load seen looking into the electrodes (ZL) is equal to R3 with a value of 10 kΩ. All benchtop tests were conducted at an RF frequency of 2.45 GHz and without voltage regulation implemented at the output. The implant generates monophasic voltage pulses in response to OOK modulation of the RF carrier with negligible delay as shown in elements A and B of FIG. 6B. which provides a higher resolution capture of an individual monophasic pulse that is delivered to a 10 kΩ load. A discharging effect can be clearly observed when the pulse width is 3 ms and its rate is determined by the time constant formed by the storage capacitor (C9) and the load at the electrodes. This discharge effect can be eliminated by increasing the value of the storage capacitor; however, in this work, the stimulation protocol comprises pulses with a width of 200 μs with a negligible discharge when using a 30 μF storage capacitor. When performing a similar test in saline, a discharge effect can be clearly observed in Element C of FIG. 6B due to the highly capacitive nature of the saline media. In this measurement, the load seen looking into the electrodes is Z L=R3∥Z eq where Zeq is the equivalent impedance of the saline media. The implant delivers several milliamps of current in saline, within the typical range for cortical stimulation applications. The output voltage was measured as a function of distance and external transmitted power as shown in element D of FIG. 6B. Each marker in the figure represents a measurement point. The implant generates up to 21 V at the maximum transmit power (30 dBm) and a distance of 25 mm to the external antenna in an air medium. The output voltage decreases as the external antenna is moved away from the implant, and the impact of increasing distance on the output voltage becomes less significant as the antenna is moved out towards the far-field region. The output voltage is also impacted by the OOK frequency at any given transmit power and distance. For any stimulation frequency below 20 Hz, the energy storage capacitors have adequate time to fully charge. However, as the frequency is increased to 300 Hz, the output voltage drops by around 50% due to insufficient charging time. For future chronic applications, it is beneficial to convert the monophasic voltage pulses into biphasic, charge-balanced pulses in order to prolong the electrodes'lifetime and avoid hydrolysis. An implementation of a charge balancing circuit, composed of a series capacitor and a shunt resistor that approximates the real impedance of tissue in parallel with the 10 kΩ resistor.

Referring to FIG. 6C, results of stimulation testing are shown. A comparison of voltage and current requirements for triggering hindlimb response in a subject between a stimulation method through the dura substitute and a direct epidural stimulation method. The top portion of FIG. 6C shows an example set up for testing. Element A of FIG. 6C shows an example closed circuit voltage and current waveforms without the dura substitute. Element B of FIG. 6C shows an example closed circuit voltage and current using dura substitute. In some embodiments, a wireless stimulator included a Rogers 6010 substrate (referred to as 6010 stimulator). The stimulator can have a similar, equivalent, or the same circuit structure as the implant 102, and it is equipped with energy storage capacitors with an identical value (30 μF). The 6010 stimulator can therefore be used to gain useful insights into the voltage and current requirements for e-dura stimulation. Two stainless steel electrodes were connected to the 6010 stimulator as shown in FIG. 6C. They were gently placed on the cortical surface and then on the dura substitute using micro-manipulators, and used to deliver electrical stimulation in these two distinct conditions. The external transmitter was placed at a distance of 30 mm away from the 6010 stimulator.

The results show that both methods can be used to trigger limb motor response. The current required in direct stimulation is around 2 mA, as shown in Element A of FIG. 6C. A larger current is needed to trigger a motor response when using the dura substitute method, and it was found to be around 6 mA, as shown in Element B of FIG. 6C. The dura substitute creates a low-resistance non-stimulation path, allowing a greater proportion of the stimulation electrode current to return to the ground electrode through the dura substitute without entering into cortex. Therefore, the effective strength of the current is reduced and a larger total current is needed to stimulate the motor neurons. Stimulation can be achieved using the proposed e-dura substitute method with at least the same reliability as the conventional stimulation method.

Referring to FIG. 6D, results of stimulation testing are shown. For example, the results include limb deflection recordings resulting from wireless electrical stimulation using the dura substitute. Element A of FIG. 6D includes limb deflection which was found by measuring the total distance traveled from the resting position. Inset in Element A is a closer view showing several tracked points and resting position. Element B of FIG. 6D shows a plot of limb deflection from one subject. Element C of FIG. 6D shows peak deflection averaged over 5 stimulation bursts recorded in n=6 subjects. Wireless stimulation triggered a motor response in all subjects (n 6) and the movement correlated with the stimulation frequency of 1 Hz during the entire recording period of 30 seconds in each subject. Limb deflection (total distance from resting limb position, Element A of FIG. 6D) was recorded for at least 30 seconds for all subject models. A capture with a period of 5 seconds showing limb deflection in subject nl is plotted in Element B of FIG. 6D. The distinctive peaks were observed when wireless stimulation was delivered at 1 Hz, and no deflection was observed in the absence of wireless transmission. The peak limb deflection, averaged over a 5-second period is plotted for all subjects in Element C of FIG. 6D. The average limb deflection measured in 6 subjects was 8.2±5.3 mm with 99% confidence level. This deflection corresponds to stimulation just above the motor neuron threshold. Further increasing the transmitted power was observed to increase the limb deflection. Note that the required stimulation current and the resulting limb deflection depend on many additional factors: the subject's anesthesia depth, any acute surgical injuries, and the exact position of the electrodes on the motor cortex can all cause variations.

The closed-circuit voltage that is required to trigger motor response was recorded while measuring limb movement. The ground electrode is placed under the skin, creating a stimulation current path that is delivered through the dura substitute. Therefore, the load seen looking into the electrodes is Z_{L}=R3|Zeq where Zeq is the equivalent impedance of the stimulation current path.

Referring to FIG. 6E, the closed-circuit measurement is shown in Element A of FIG. 6E for a period lasting a full burst and in Element B of FIG. 6E which provides a higher resolution capture of a single monophasic voltage pulse for subject nl. Similar to current measurements in saline media, the voltage pulses exhibit a discharge effect when testing in vivo.

The open circuit voltage was found by disconnecting the ground electrode while maintaining the implant's placement and distance to the external antenna and keeping the RF transmit power and stimulation parameters unchanged. By removing the ground electrode, the stimulation current path is open and its equivalent impedance is Zeq is approximately infinite and the load looking into the electrodes is ZL=10 kΩ. A recording of the open circuit voltage is shown in Elements C and D of FIG. 6E. The peak closed circuit and open circuit voltage are taken for n=6 subjects and are shown in Elements E and F of FIG. 6E, respectively. The average closed-circuit voltage is 4.57±1.13 V with 99% confidence level. Comparing the closed-circuit voltage with the current measurements in FIG. 6E facilities an approximate value of ZL=R3∥Zeq which is the equivalent impedance of the stimulation current path in parallel with the 10 kΩ load resistor at 2 kΩ. These results show good consistency with the data provided in where the impedance magnitude of a collagen solution was found to range between 1 kΩ and 3 kΩ at a frequency of 100 Hz, depending on the concentration of collagen in the solution. This estimation is useful when determining the value of the series capacitor that is used for charge balancing when testing the implant in a chronic setting. The average open circuit voltage is 7.47±1.11 V with a confidence level of 99%.

Referring to FIG. 6F, Element A shows measured voltage and current waveforms measured in vivo. Ten pulses with a width of 200 μs at a frequency of 100 Hz are delivered at 1 Hz. Element B of FIG. 6F shows resulting hindlimb movement observed at 1 Hz. The implant triggered hindlimb movement. In this example, a cranial window with dimensions of 3×7 mm² was opened in a subject's skull under anesthesia. The dura was kept intact during the experiment. The device was placed gently on the motor cortex as shown in to deliver monophasic stimulation pulses. The silicone rubber pedestal allows the device to remain flat on the surface of the skull while making soft contact with the cortical surface. The ground wire was placed under the skin, and another wire was connected to the positive electrode to take measurements. An external transmitter was used to generate a pulse train composed of ten pulses at a frequency of 100 Hz with a pulse width of 200 μs. The pulse train was delivered at a frequency of 1 Hz. The wireless stimulator was positioned at a distance of around 20 mm away from the external transmitter.

Element A of FIG. 6F shows the recorded closed circuit voltage and current signals, respectively, during the onset of stimulation when hindlimb movement was observed, indicating the structure of the stimulation pulses. The implant generates monophasic pulses with an amplitude of around 8 V and it injects a current of 2 mA into the cortex. The current was found by measuring the voltage drop across a 10Ω series sensing resistor.

Element B of FIG. 6F shows a plot of the hindlimb movement in millimeters, measured from a video recording using analysis software (Tracker video analysis and modeling tool). The hindlimb deflections show a clear correlation with motor cortex stimulation. These results demonstrate that the wireless implant can provide an adequate voltage and current to trigger a motor neuron response and the movement occurs on the onset of stimulation at a frequency of 1 Hz. Additionally, this example shows that the implant can generate any stimulation pattern, ranging from a single monophasic pulse that can be used for cardiac stimulation to a more complex pulse train with a specific frequency, pulse width, and interpulse spacing that is used for cortical stimulation.

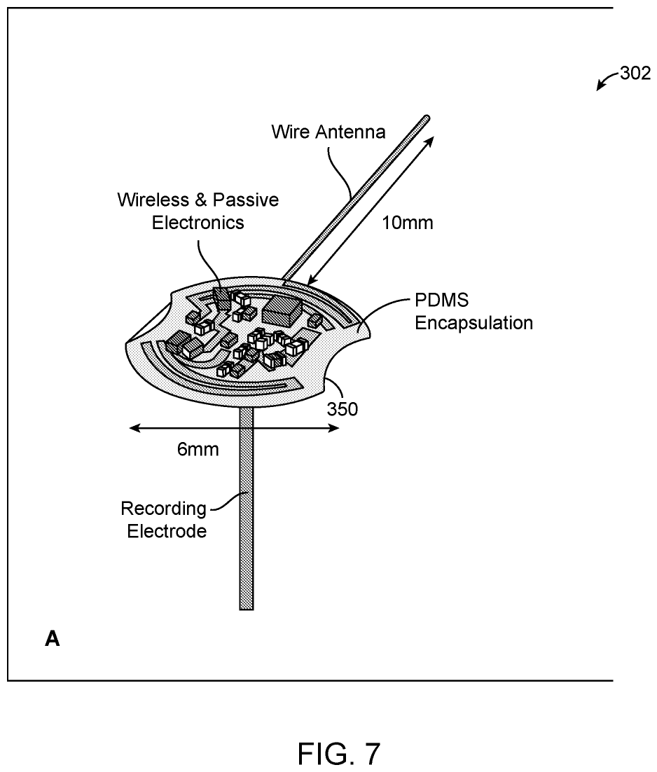

Referring to FIG. 7, an example implant 302 is shown. The implant 302 can share the features of implants 102, 202 described above. For example, the implant 302 includes a flexible substrate 350 that houses the wireless and passive electronics (e.g., such as the components shown and described in FIGS. 2A and 2B). The implant 302 can include an artifact reduction mechanism, and a depth electrode that can be used for measuring seizure inside the brain tissue.

Referring to FIGS. 8A-B, the implant (e.g., implant 102, 202) is fabricated following a series of steps. In some embodiments, the steps are shown in FIGS. 8A-B.

The implant is fabricated on a silicon wafer in a low temperature process. The following exemplary steps can be followed.

Element A of FIG. 8A shows PDMS is spin-coated on a 4-inch silicon wafer at 200 rpm and 1:10 curing agent ratio. It is soft-baked at 75° C. for 24 hours. Element B of FIG. 8A shows the wafer is coated with the parylene interface layer (Dimer-C, 8 μm) using the SCS Parylene Coater. Element C of FIG. 8A shows the wafer is treated with oxygen plasma for 3 minutes to clean the parylene surface and enhance the adhesion of gold. Element D of FIG. 8A shows a layer of thin gold films (300 nm) was deposited onto the wafer using RF sputtering (EMITECH K675X). Element E of FIG. 8A shows the wafer is spin-coated with HMDS at 2000 rpm and soft-baked at 100° C. for 1 minute. It was then spin-coated with AZ 4330 positive photoresist at 2000 rpm and then soft-baked at 100° C. for 1 minute. The wafer was then exposed to UV light for 30 seconds through a mylar photomask using a mask aligner (OAI808). Element F of FIG. 8A shows the photoresist is patterned by submerging the wafer in MIF 300 developer for 90 seconds. Element G of FIG. 8A shows the wafer is submerged in gold etchant (GE-8148) for 30 seconds to obtain the circuit features. Element H of FIG. 8A shows the surface mount components and the antenna are attached to the gold tracks using conductive silver epoxy (12642-14, Electro Microscopic Science). The wafer is soft baked at 100° C. for 5 minutes. Element I of FIG. 8A shows the implant is cut and removed from the wafer for further processing. The electronics and the positive electrode are integrated at top and bottom layers, respectively. Element J of FIG. 8A shows a parylene encapsulation layer (Dimer-C, 2 μm) is then deposited using the SCS Parylene Coater, leaving the tip of the positive electrode exposed.

FIG. 8B shows exemplary steps for integration of a positive electrode into the implant of FIG. 8A. Element A of FIG. 8B shows a silicone rubber pedestal (Ecoflex, 00-30) with dimensions of 2×2×1 mm³ is attached to the bottom of the device using silicone adhesive (MED 1-4013, Avantor). The silicone adhesive is cured at 70° C. for 20 minutes. Element B of FIG. 8B shows a micro-drill (0.3 mm, LPKF) is used to make a through-hole at the location of the positive electrode by manually rotating at around 30 rpm. A stage made of clear resin with a 5-mm wide through-hole was 3D printed (e.g., form3, Formlabs) and used to assist in handling the implant during the drilling process. Element C of FIG. 8B shows a 127 μm wire is passed to form a VIA that connects the top layer to the bottom layer. It is attached to the top and bottom layers using conductive silver epoxy. A disk-shaped, gold-plated brass electrode (Series 815-22, Mill-Max Manufacturing) with a diameter of 1.2 mm is connected to the stainless steel VIA at the bottom using conductive silver epoxy. Element D of FIG. 8B shows silicone adhesive is used to insulate the electrode, leaving the tip exposed. It is cured at 70° C. for 20 minutes. Element E of FIG. 8B shows a side view of the implant showing the topology of the positive electrode. Element F of FIG. 8B shows a bottom view of the implant showing the positive electrode. Element G of FIG. 8B shows the silicone elastomer pedestal that is used in the subject in vivo setup. Element H of FIG. 8B shows a photograph of the disk electrode before integration with the implant.

In some aspects, the implant includes Polydimethylsiloxane (PDMS) (εr=3) forms the main substrate (100 μm) on top of which gold traces interconnect the implant circuit. A parylene interface layer (8 μm) is added between the PDMS and the gold layer to enhance the adhesion of gold to the PDMS substrate while maintaining flexibility and transparency. This combination of two dielectric materials facilitates low permeability to moisture (Dimer C). A parylene passivation layer (2 μm) is added to protect and electrically isolate the electronics. A soft silicone elastomer pedestal is then attached to the bottom of the implant, underneath the positive electrode to fill the gap between the skull and the dura substitute for testing. The pedestal has a thickness that is similar to the subject skull (e.g., 1 mm) and it allows the implant to be positioned on the bone during in vivo testing. Electrical stimulation is delivered from the top layer through a stainless steel VIA to a gold-plated disk electrode with a diameter of 1.2 mm that is attached using silver epoxy to the silicone elastomer pedestal. In some aspects, the assembly process may not require the silicone elastomer if the implant can fit on the dura substitute. Instead, only a VIA and a similar electrode with possibly a larger diameter can be used. The antenna is formed of two coated and flexible stainless wires each with exemplary lengths of 25 mm and a diameter of 127 μm. Measurement wires are connected to the implant for data acquisition. Combining soft dielectric materials with gold, a malleable metal, results in robust tolerance to bending. The implant maintains transparency, allowing clear observation of the text underneath. These mechanical properties make the implant a suitable tool for biomedical applications where flexibility and small thickness are paramount to avoid complications. Additionally, maintaining transparency and miniaturized overall dimensions ease handling, in vivo aligning the implant to target a specific cortical region during the surgery.

Referring to FIG. 8C, an example charge balancing circuit for extending the lifetime of electrodes and results are shown. For example, Element A of FIG. 8C shows the equivalent resistor can be found by comparing the closed circuit voltage with the measured current. Element B of FIG. 8C shows capacitive charge balancing using a 50 nF capacitor.

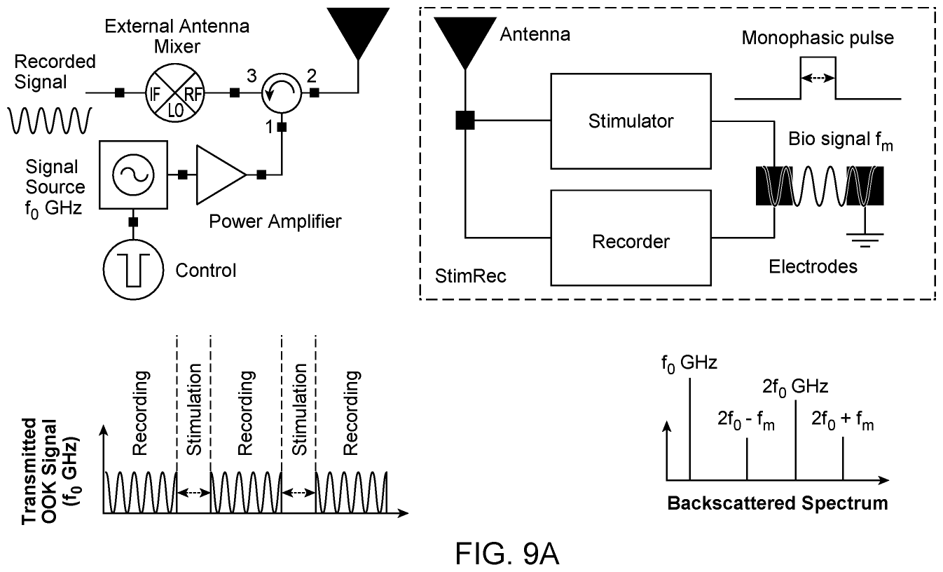

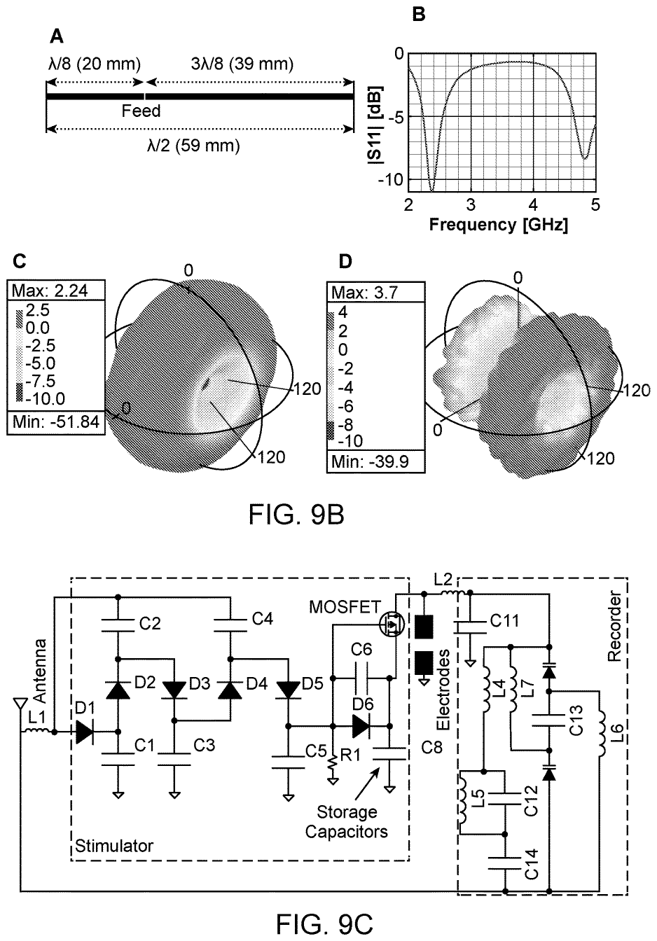

Referring to FIG. 9A, a block diagram shows the stimulator (e.g., implant) and the recorder (e.g., external device). In some embodiments, the stimulator and the recorder generate monophasic voltage pulses and acquire biosignals through RF backscattering.

The implant includes a dual-band antenna that operates at f₀ and 2f₀. At the antenna's output, the stimulator includes a rectifier to convert wireless power into DC voltage, a switch to produce the stimulation pulses, and several energy storage capacitors. Monophasic voltage pulses appear at the electrodes when the switch is turned ON. In some embodiments, the same pair of electrodes acquire a signal at a frequency fₘ. The recorder includes an array of varactor diodes that generate the third-order non-linear mixing products (2f₀±fₘ). The dual-band antenna backscatters the third-order mixing products to be detected and demodulated by the external transceiver. The external OOK signal facilitates arbitrary switching between stimulation and recording. Charging the storage capacitors, backscattering, and recording occur within the OOK control signal ON intervals. During this period, the external transceiver detects, amplifies, and demodulates the backscattered signal at 2fₒ±fₘ to obtain the recorded signal at the frequency fm. In some embodiments, stimulation occurs in the OOK OFF intervals. The frequency and pulse width of the monophasic voltage pulses are controlled by modifying the frequency and duty cycle of the OOK signal.

Referring to FIG. 9B, an off-center-fed dual-band dipole antenna is shown with associated results. A dipole antenna operating at a frequency of fₒ can exhibit odd harmonics; however, shifting the feed position by a quarter length can generate even harmonics. The implant can utilize this property to receive the RF carrier at fₒ and backscatter the third-order harmonic at 2f₀+fₘ. An off-center-fed, half-wavelength dipole antenna can include the dimensions shown in Element A of FIG. 9B. The reflection coefficient simulation results, presented in Element B of FIG. 9B show that the antenna operates at fₒ=2.4 GHz and 2fₒ=4.8 GHz. These characteristics facilitate the antenna's reception of the carrier signal at 2.4 GHz and backscatter the third-order product at 4.8 GHz. The radiation pattern simulation results show that the antenna exhibits an omnidirectional radiation pattern at 2.4 GHz with a maximum realized gain of 2.24 dBi to a 50 Ohm source that occurs at the center of the dipole, as shown in Element C of FIG. 9B. On the other hand, the antenna exhibits an omnidirectional radiation pattern with a center null and a maximum realized gain of 3.7 dBi that occurs at the center of the long dipole arm.

Referring to FIG. 9C, an example circuit diagram of the stimulation and recording system is shown. The stimulator and recorder are connected to the antenna through a matching element inductor (L1) to compensate for the capacitive input impedance. The stimulator comprises a Schottky diode voltage multiplier (DI-6) (e.g., JDH2S02SL, Toshiba) to rectify the wireless signal, followed by storage capacitors (C8) (30 μF) to accumulate the harvested voltage, and a MOSFET switch (e.g., PMZ320UPEYL, Nexperia) to control the delivery of voltage pulses. An RC timer (C5 R1) at the gate discharges during the OOK OFF intervals, turning the MOSFET ON to deliver a monophasic voltage pulse from the storage capacitors. The recorder contains two varactor diodes (e.g., MA46H 120, Macom) that receive the 2.4 GHz wireless signal and generate the mixing products. The use of multiple varactor diodes enhances the recorder's sensitivity. Moreover, an RF choke (L2) is placed between the recorder and the MOSFET to block the RF signal from the drain. An RF shorting capacitor (C6) is connected in the low-frequency path to prohibit any RF signals from appearing at the transistor.

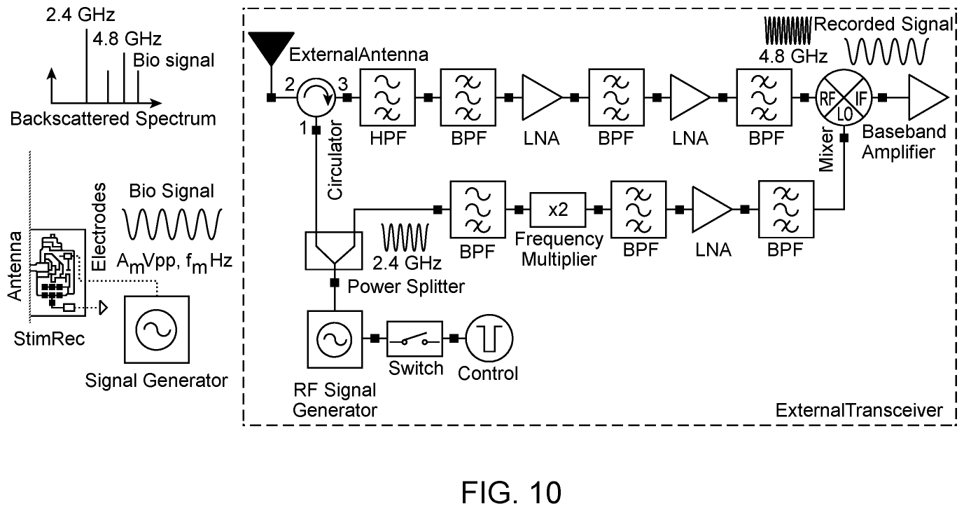

Referring to FIG. 10, the stimulator and recording system can be implemented using the setup shown in FIG. 10. For example, an external transceiver that contains a zero-IF receiver is tuned to the third-order harmonic signal at 4.8 GHz. A direct conversion mixer (e.g., ZX0S-762H-S+, Mini-Circuits) recovers the emulated biosignal at the IF port. A baseband amplifier (e.g., SR560, Stanford) boosts and filters the baseband signal. It provides a gain of 200 and a bandpass filter to reject external noise. The external transceiver is equipped with an antenna (e.g., A10194, Antenova) with dimensions of 10×10 mm² and gain of 1.8 and 4.1 dBi at 2.4 GHz and 4.8 GHz, respectively. An RF signal generator (e.g., 5009, Valon Technology) is used to obtain the transmitted signal at 2.4 GHz. The local oscillator (LO) at 4.8 GHz is obtained from a frequency doubler (e.g., ZX90-2-36-S+, Mini-Circuits) connected to a −3-dB power splitter (e.g., ZAPD-4-S+, Mini-Circuits). A power amplifier with a gain of 25 dB (MPA- 24-20, RF bay) is used to boost the transmitted signal level. OOK modulation is obtained through an arbitrary signal generator (e.g., E4432B, Agilent) and an RF switch (e.g., ZFSWA2-63DR+, Mini-circuits). The backscattered spectrum is delivered to a filtering stage to eliminate the 2.4 GHz signal and obtain the third-order mixing product at the input of the low-noise amplifier (LNA) (e.g., 4560, RF Bay). The external transceiver's antenna is aligned to the center of the implant's long dipole arm to obtain a balanced gain at 2.4 and 4.8 GH z.

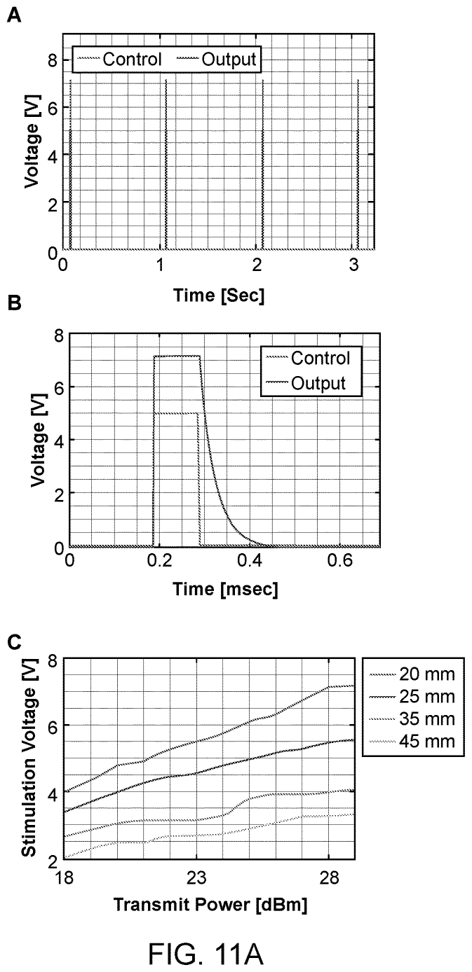

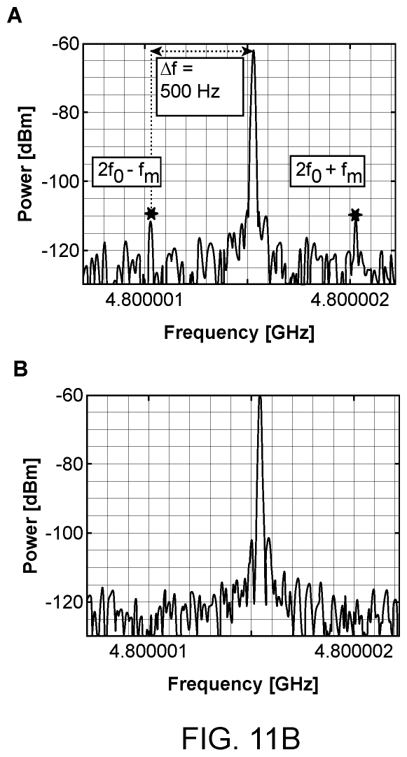

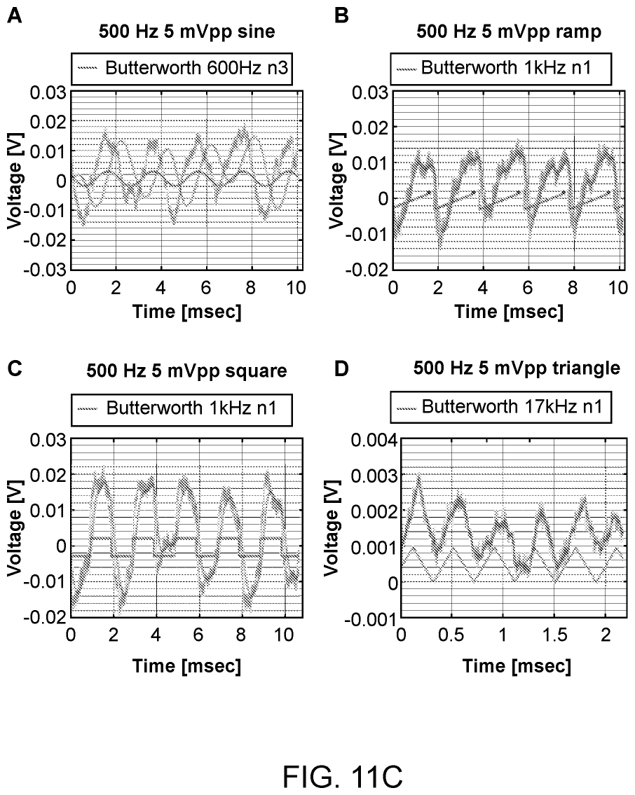

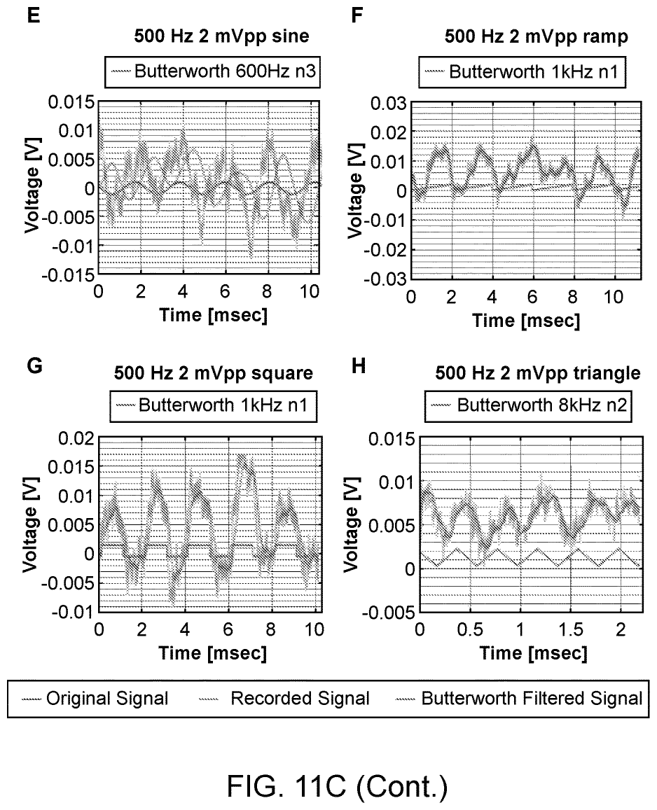

Referring to FIG. 11A, results of stimulation testing are shown. For example, Element A of FIG. 11A shows stimulation output voltage as a function of the OOK control signal for a stimulation frequency of 1 Hz. The stimulation output voltage was characterized as a function of transmitted RF power, distance to the external transceiver, and stimulation frequency. Element A of FIG. 11 A shows that the implant generates monophasic voltage pulses in response to an OOK control signal with negligible delay and matching stimulation frequency (1 Hz). Element B of FIG. 11A shows a higher resolution capture indicating that the output voltage has a similar width as the control signal (100 μs). The output voltage as a function of RF power and distance is shown in Element C of FIG. 11A. A maximum output of 7.2 V can be at a distance of 20 mm. The device can generate 2 V at a distance of 45 mm and transmit power of 18 dBm.

Referring to FIG. 11B, results of recording testing are shown. For example, Element A of FIG. 11B shows the measured wireless spectrum in the presence of a signal at the implant's electrodes. For example, Element B of FIG. 11B shows the measured wireless spectrum in the absence of a signal at the implant's electrodes.

Wireless recording can be verified by measuring the backscattered RF spectrum from an implant that was fabricated on a Rogers 4003 substrate. A sine wave with an amplitude of 20 mVₚₚ is applied at a frequency fₘ=500 Hz at the implant's electrodes. The external transceiver receives the third-order mixing product. A spectrum analyzer (e.g., N9913A, Keysight) is connected at the receiving port (circulator port 3 of FIG. 10), and the third-order mixing product is measured in the presence and absence of the biosignal. The measurement was performed with the transceiver placed at 25 mm from the implant. Element A of FIG. 11B shows the measured spectrum in the presence of the biosignal exhibiting the 4.8 GHz harmonic and the biosignal frequency components at 2fₒ±fₘ where fₘ=500 Hz. The magnitude of the recorded biosignal was around −111 dBm. Element B of FIG. 11B shows the measured spectrum in the absence of the biosignal, leading to the lack of the third order mixing product, while the 4.8 GHz harmonic can still be observed. These results confirm that the implant performs harmonic mixing of the 2.4 GHz RF signal with the biosignal at the electrodes. The recorded signal in the baseband is examined at a transceiver distance of 20 mm at the IF port (FIG. 10).

Referring to FIG. 11C, sine, square, ramp, and triangle waves with varying amplitudes are applied at the implant's electrodes at a frequency of 500 Hz. and are presented as the original signal. The original signals are applied with an amplitude of 5 mVpp and 2 mVpp in Elements A-D and E-H of FIG. 11C, respectively. The signals recorded at the output of the baseband amplifier are shown in each subfigure. The recorded signal matches the shape and frequency of the original signal with noticeable high-frequency noise that distorts the recorded signals. In addition, the peaks of the square and ramp waves are distorted due to baseband filtering. The integrity of the recorded signals deteriorates as the amplitude of the original signals is decreased from 5 mVpp to 2 mVpp. Therefore, lowpass Butterworth filtering is applied to the recorded signals with varying order (n) and cutoff frequency, which enables the recovery of the emulated biosignals with an amplitude as low as 2 mVpp. The recorded signals become indistinguishable as the amplitude of the original signal is reduced below 2 mVpp. These results demonstrate that the implant can record an emulated biosignal with a sensitivity of 2 mVpp.

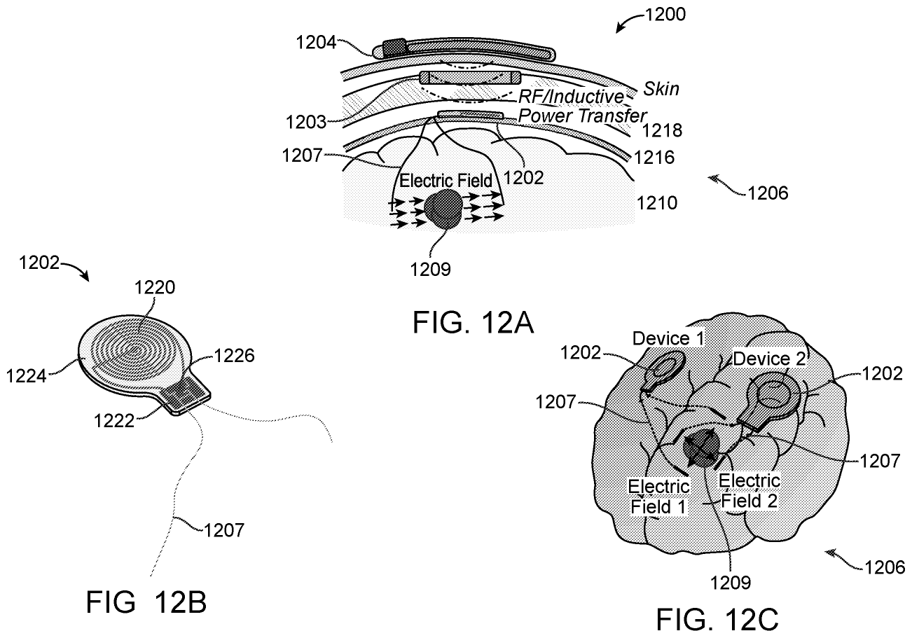

Referring to FIG. 12A, a schematic view of an example treatment system 1200 is shown. For example, the treatment system 1200 can include implant 1202 and an external device 1204. The implant 1202 and external device 1204 can share features with the implants 102, 202 and external devices 108, 208 described herein.

The implant 1202 can be a miniaturized, wireless, and passive device implanted in the head 1206 of a subject. The implant 1202 can facilitate the treatment of tumors by delivering an electric field (e.g., a tumor treatment field “TTF”) through subdural lead electrodes.

The positioning of the implant 1202 within the layers of the head 1206 of the subject is illustrated. In some aspects, implant 1202 can be directly positioned on the dura mater 1216 or dural substitutes. The implant 1202 has a low profile that facilitates the ability of the implant 1202 to contour to the curved surface of the dura 1216, ensuring a stable contact between one or more electrodes of the implant 1202 and the tissue of the dura 1216. The implant 1202 can be implanted under the skull 1218 for long-term neural recording and stimulating operations without intracranial wiring.

The placement of the implant 1202 facilitates several advantages, including continuous use, reduction of migration, reduction of infection, reduction of other adverse events, increased spatial and temporal sensitivity. For example, the implant 1202 can be used continuously. The placement of the implant 1202 between the bone and the dura reduces the risk of infection, the risk of migration, and the risk of other adverse events. The implant 1202 can have increased temporal and spatial sensitivity that is facilitated by the direct access to the cortical surface. The increased temporal and spatial sensitivity facilitates high precision stimulation or monitoring by the implant 1202.

The implant 1202 can be implanted on the dura 1216 and is powered by an external transceiver 1204 mounted on the scalp of the patient. Alternatively, the implant 1202 can also be integrated with a dura graft and be used as a substitute for damaged dura tissue. In a single-channel embodiment, a pair of lead electrodes can be inserted into the brain tissue or the ventricles to deliver an electrical field across a target tumor 1209. The exact placement location of the lead electrodes 1207 can be determined by physicians to achieve the best treatment outcome. The external device 1204 can be fixed on the skin and aligned to the implant 1202 with help of a magnetic ring 1203 buried inside the skull 1218.

Referring to FIG. 12B, the implant 1202 is illustrated removed from the head 1206 of the subject. The implant 1202 can be a single-channel implant. The implant 1202 can include a wireless element 1220 for receiving power from the external transceiver 1204. This wireless element 1220 can be in the form of an inductive coil or an antenna. The implant 1202 can include a flexible printed circuit board 1222 (PCB) containing electronic components and circuits for generating the TTF signal. The substrate of the PCB 1222 can be any flexible and biocompatible material, including polyimide, parylene, PET (polyethylene terephthalate), PC (Polycarbonate), or other similar materials. The substrate can also be any biocompatible, flexible, and stretchable elastomer, such as silicone rubber or PDMS. Different circuit structures may be used depending on the applications.

The implant 1202 can include a pair of lead electrodes 1207 for delivering a sine wave (or other periodic waveform) TTF field. In some embodiments, the lead electrodes 1207 can be single-thread wires coated with an insulation layer, or coaxial cables in which the signal will be delivered through the center wires. The material for the electrodes 1207 can be any biocompatible metal or conductor, including stainless steel, platinum, gold, titanium, etc. The implant 1202 can include a flexible and biocompatible encapsulation 1224 that protects and insulates the flexible circuits and the wireless element. The implant 1202 can also include passive electronics 1226 that facilitate communication between the implant 1202 and the external device 1204.

Referring to FIG. 12C, two implants 1202 can be implanted into the head 1206 of the subject. The arrangement shown in FIG. 12C facilitates the placement of two single-channel implants 1202 for generating two directional alternating electrical fields. The two directional alternating electrical fields each pass through the tumor 1209 in different directions. For example, each set of electrodes 1207 can be positioned around opposing sides of the tumor 1209 to direct the electrical fields across the tumor 1209 in different directions. The two implants 1202 can be positioned for generating perpendicular TTF fields. The implants 1202 can be placed on the left and right hemispheres respectively. The lead electrodes 1207 of each implant 1202 will be inserted into the brain tissue (or ventricle) and aligned in two perpendicular directions surrounding the target tumor 1209. Each of the implants 1202 can be powered by a separate external transceiver 1204 placed on the scalp of the patient. For effective treatment of tumor 1209, each of the implants 1202 will be activated in turn to generate alternating electrical fields in the corresponding directions.

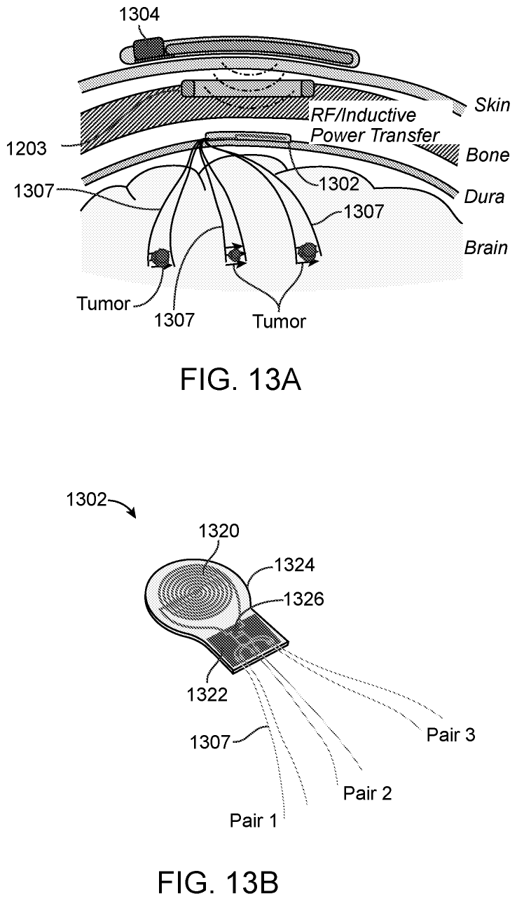

Referring to FIGS. 13A and 13B, an example implant 1302 with multiple channels is shown. In some embodiments, the implant 1302 is a multichannel wireless TTF implant containing multiple pairs of leads 1307 of electrodes. These electrode pairs 1307 can be configured to deliver the TTF field synchronously or alternately. Compared with the single-channel device (e.g., implant 1202), the implant 1302 can deliver stronger fields over a larger area inside the brain tissue, as shown in FIG. 13A. Example embodiments of the implant 1302 can be implemented when the tumor has spread to multiple locations and one channel device does not provide efficient coverage of the TTF fields. In some embodiments, the implant 1302 can be implemented when the tumor at a specific location is large and calls for a strong TTF field to have treatment effect. The implant 1302 can also be configured to generate TTF fields at each electrode pair in turn. For this configuration, a single implant 1302 multichannel can generate the two perpendicular electrical fields.

The implants 1202, 1302 can be positioned in a variety of locations, each having particular advantages. For example, the implants 1202, 1302 can be extracranial: under the scalp, above the skull. This placement allows the easiest surgery. Optionally, an extracranial electrode may be paired with an intracranial electrode (any position) to prevent scalp shunting: as long as at least one electrode of the pair is below the skull, the current will be constrained to travel through the skull and not only through the scalp. Such an extracranial-to-intracranial pair may benefit from the resistance of the skull, because the skull could serve to spread the current over a wider area and thereby distribute the therapy more uniformly across the brain.

In some embodiments, the implants 1202, 1302 can be epidural: below the skull, above the meningeal dura. This circumvents the skull. The dura is still a moderately resistive barrier, and thereby could achieve a similar current-spreading effect as the skull, which could be utilized to allow higher current density from the electrode without exceeding damage thresholds of current density within brain tissue. That is, the current-spreading effect may allow higher current from smaller electrodes without excessive peak current in the tissue directly below the electrode.

In some embodiments, the implants 1202, 1302 can be subdural: below the dura, on the cortical surface.

In some embodiments, the implants 1202, 1302 can be parenchymal: within the brain tissue. Such penetrating electrodes could be similar to depth electrodes used for stereoelectroencephalography, or similar to electrodes used to deliver deep brain stimulation. This could include an array of many electrodes.

In some embodiments, the implants 1202, 1302 can be ventricular: inserted through the brain into one or more ventricles, cisterns, or other spaces containing cerebrospinal fluid. CSF has a very low electrical resistance, and therefore could serve to spread the TTF current across a wider area to achieve more uniform therapy without excessive peak intensity near the electrode.

In some embodiments, the implants 1202, 1302 can be intravenous: inserted into an artery or vein within or above the brain. Similar to ventricular placement, the low-resistance blood could serve to distribute current over a wider area.

In some embodiments, the electric fields generated by the implants 1202, 1302 can have a variety of arrangements. For example, TTF is less effective when the field direction is perpendicular (orthogonal) to the axis of the mitotic spindle of the dividing cells. For this reason, TTF uses at least two different field orientations so that at least one of the fields will be effective on all dividing cells (e.g., in some aspects, no axis can be perpendicular to both field directions).

An implanted TTF system (e.g., using implants 1202, 1302) may use various electrode arrangements. An example embodiment includes 4 electrodes, 2 separate pairs. Denoting electrodes as A, B, C, D, the pairs of two electrodes would be denoted (A, B) and (C, D). Each electrode may be a single continuous structure or can be an array of electrically-connected electrodes. Another example includes 3 electrodes, 2 overlapping pairs with 1 common: (A, C), (B, C). Another example includes 3 electrodes, 3 pairs. (A, B), (B, C), and (C, A).

In some embodiments, the electrodes 1207, 1307 can be a flat sheet, or a wire, or any other shape. The shape can include a high surface area or can be shaped for easy insertion. An electrode may have one or more insulated portions. An electrode can include a controllable array of contacts, where individual contacts may be used or unused. Individual contacts may be selected in order to shape the field to achieve optimal therapy. An electrode can be platinum, stainless steel, titanium, or other conductive material. An electrode may be capacitive. As one example, a capacitive electrode may have a thin layer of passivation, such as titanium nitride.

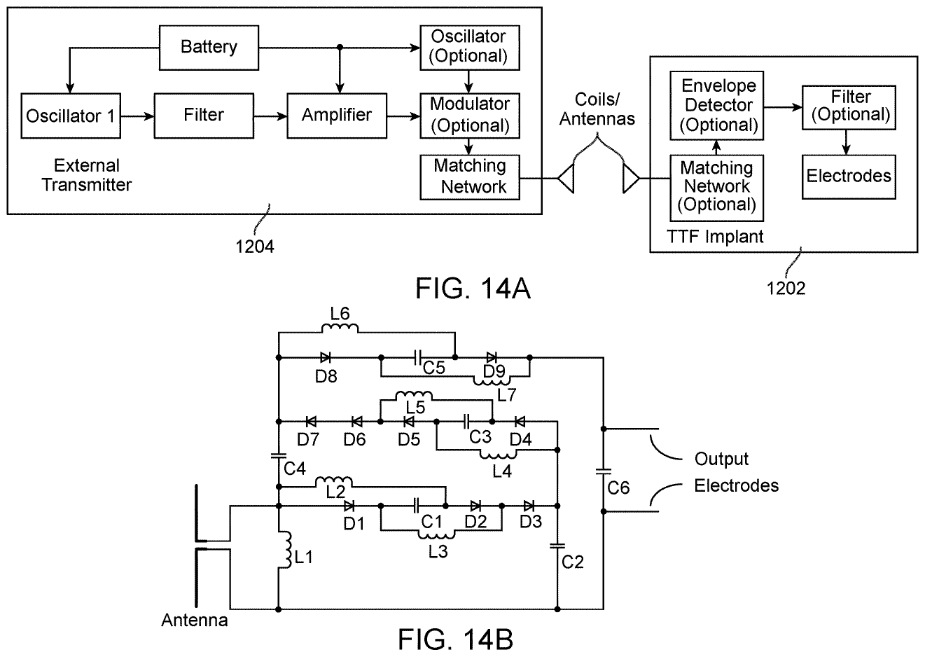

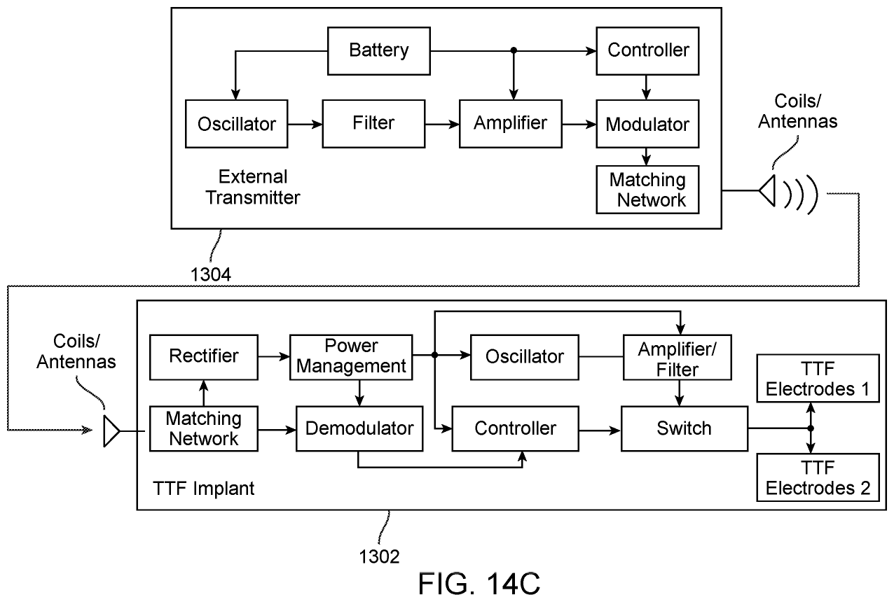

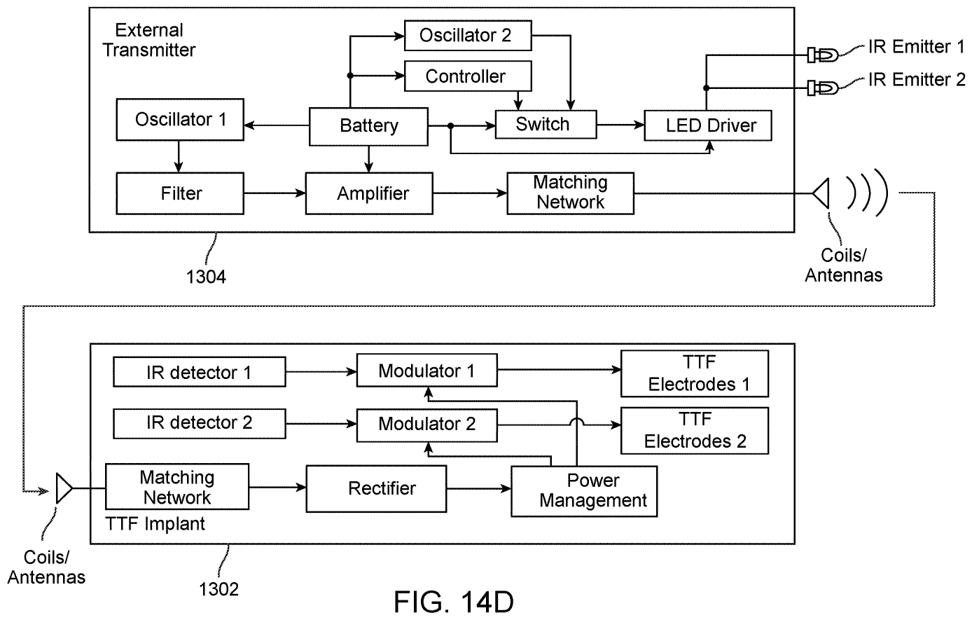

Referring to FIG. 14A, an example block diagram showing the structure of a single-channel TTF system. FIG. 14A shows the structure of a single-channel TTF system, which includes an external transmitter 1204 and a wireless TTF implant 1202. The external transmitter 1204 powers the TTF implant 1202 through the wireless elements (coils or antennas).

The external transmitter 1204 includes the main oscillator (oscillator 1) for generating a carrier signal of a fixed frequency. This oscillator can be designed using any analog or digital oscillator circuit structure, such as ring oscillators, LC oscillators (Colpitts or Hartley), RC oscillators, crystal oscillators, Schmitt trigger oscillators, 555 timers, etc. The frequency of the carrier signal can be the same as the target TTF signal (100-200 KHz) or much higher (MHz-GHz). In the simplest structure, the oscillator frequency will be the same as the TTF signal. The external transmitter 1204 includes one or multiple filters for filtering out unwanted noise in the carrier signal. The filter can be a band-pass filter of any type, such as butterworth, chebyshev, or others. It can be made from passive components such as resistors, inductors, or capacitors. Active components can be included to achieve a higher gain. The external transmitter 1204 includes an amplifier to increase the power of the carrier signal. If the carrier signal is in the low-frequency range (100-200 KHz), this amplifier can be a power amplifier of any type (Class A, B, AB, or C). When the carrier frequency is in the high-frequency range (MHz-GHz) the amplifier can be designed as an RF power amplifier. The external transmitter 1204 includes a matching network to achieve impedance matching between the circuit and the wireless power transferring element. The external transmitter 1204 includes a wireless element. For low-frequency carrier signals, this element can be a coil. For high frequency signals, it can be either a coil or an antenna. The external transmitter 1204 includes a battery or similar stored energy source for providing a DC power supply for the external transmitter.

If the carrier signal generated by the main oscillator (Oscillator 1) has a frequency much higher than the TTF signal, a second oscillator (Oscillator 2) can be implemented to create a second sine wave signal which has the same frequency as the TTF field. This signal can be used to modulate the carrier signal.

The external transmitter 1204 includes a modulator to mix the sine wave signals generated by the two oscillators. The modulator can be designed as a single or double-balanced mixer. The mixing components can be either diodes (such as the diode bridge circuit) or transistors (such as the gilbert cell). In the simplest case, the mixer can also be implemented as one single diode or varactor to save power.

The TTF implant 1202 will harvest energy from the external transmitter 1204 and generate a TTF electrical field of 100-200 Khz at the output of the lead electrodes. The TTF implant 1202 can include a wireless element for receiving wireless power. As described previously, this can be a coil or an antenna. The TTF implant 1202 can include a matching network including inductors and capacitors to achieve impedance matching for maximum power transfer. If the transmitted carrier signal has the same frequency as the TTF field (100-200 KHz), the lead electrodes can be connected at the output of the matching network. In this configuration, the TTF field can be generated through inductive coupling, no additional circuit is required. If the transmitted carrier signal is a high-frequency signal modulated by the TTF signal, an envelope detector can be used to extract the target TTF signal. The envelope detector can be implemented by diodes or transistors. The structure of the envelope detector can be a rectifier containing a single diode and capacitor. Additional diodes or rectifiers can be added to achieve a voltage-multiplying function. The TTF implant 1202 can include one or multiple filters for selecting the target TTF signal and filtering out the unwanted noise. The filters can be made from passive components such as resistors, inductors, and capacitors. The TTF implant 1202 can include lead electrodes for delivering the TTF field into the brain tissue.

Referring to FIG. 14B, an example envelope detector circuit utilizing multiple diodes to increase the output voltage across the electrodes Multi-channel system based on digital control.

Referring to FIG. 14C, a block diagram showing the structure of the multichannel TTF system based on digital control. FIG. 14C shows the structure of the wireless TTF system utilizing digital control to achieve multichannel operations. Here, the external transmitter 1304 is responsible for both powering the implant 1302 and giving digital commands to the implant 1302 to achieve multichannel selection. This can be accomplished by transmitting an RF signal encoded with digital commands through the wireless elements (coils or antenna). The components of the external transmitter 1304 are similar to the single-channel system, as shown in FIG. 14A. Some embodiments include differences. The oscillator generates a carrier sine wave signal which is in the high-frequency range (MHz-GHz). A controller outputs digital commands signal to the modulator to modulate the carrier signal. The controller can be implemented by a microcontroller IC. It can even be as simple as a digital oscillator such as a 555 timer, in which case one digital pulse will be the channel sweep command.

The multichannel TTF implant 1302 can harvest energy from the wireless signal and extract the digital commands. The TTF field can be generated by the implant device 1302 and delivered to the target channel specified by the digital commands. The multichannel TTF implant 1302 can include a wireless element for receiving wireless power. The implant 1302 can include a matching network for achieving the maximum power transfer. The implant 1302 can include a rectifier for converting the high-frequency AC signal into a DC voltage. The circuit structure of the rectifier is similar to the envelope detector mentioned above. Multiple diodes can also be used to increase the output voltage. The implant 1302 can include a power management circuit for accumulating electrical charges and providing power supply to the digital circuit and active components of the TTF implant. A PMU IC chip or a large capacitor can be used. The implant 1302 can include a demodulator to extract the digital commands from the wireless signal. The demodulator can be realized by a simple rectifier followed by a comparator circuit. The implant 1302 can include an oscillator for generating the sine wave TTF signal (100-200 KHz). The implant 1302 can include an amplifier for increasing the power of the TTF signal. Like the implant 1202, the amplifier can be any type of power amplifier (Class A, B, AB, or C). The implant 1302 can include a digital controller to process the command signals and generate corresponding switching signals. This can be a microcontroller IC that is programmed to generate channel selection output at its IO ports. If the controller in the external transmitter is a digital oscillator (such as a 555 timer), the command signals can be a single pulse indicating the channel sweep action. In this example, the controller can be a shift register that shifts its output one bit to the right after receiving each command pulse. The implant 1302 can include a series of switches to turn on/off the TTF channel after receiving the switching signal generated by the controller. The switches can be realized by a transistor, any type of analog switch, or MUX. The implant 1302 can include lead electrodes for delivering the TTF field into the brain tissue. The implant 1302 can include a multi-channel system utilizing additional communication media.

Referring to FIG. 14D, a block diagram showing the structure of the multichannel TTF system including IR energy as the second wireless communication media. The multichannel wireless TTF system can also include a second wireless communication media such as infrared (IR) light or ultrasound. In some embodiments, the incorporation of a second wireless communication medica can simplify the circuit and reduce total power consumption of the implant 1302. FIG. 14D shows an example of the multichannel TTF system utilizing IR energy as the second wireless communication method. The external transmitter powers the TTF implant 1302 through electromagnetic energy. Channel selection can be achieved by selectively emitting IR signals at different wavelengths.

The external transmitter 1304 can include the main oscillator (oscillator 1) for generating the high frequency sine wave carrier signal. The external transmitter 1304 can include one or multiple filters for filtering out unwanted noise in the carrier signal. The external transmitter 1304 can include an amplifier to increase the power of the carrier signal. The external transmitter 1304 can include a matching network to achieve impedance matching between the circuit and the wireless power transferring element. The external transmitter 1304 can include a wireless element. It can be either a coil or an antenna. The external transmitter 1304 can include a battery for providing a DC power supply for the external transmitter. The external transmitter 1304 can include a second oscillator (oscillator 2) for generating a sine wave signal that has the same frequency as the target TTF signal (100-200 KHz). This signal will be used to modulate the IR light. The external transmitter 1304 can include a controller for generating control signals to selectively turn on/off the IR LED emitters. The controller can be a microcontroller IC that is programmed to output the switching signals at its IO ports. The controller can also be a digital oscillator followed by a shift register which shifts its output ports one bit after each pulse. If two channels are used, a digital oscillator can be used as the controller. The external transmitter 1304 can include a series of switches to select which LED to be turned on. Each LED corresponds to a different channel in the implant TTF device. The switches can be realized by a simple transistor, any type of analog switch, or MUX. The external transmitter 1304 can include a LED driver circuit to drive the LED emitter with a modulated amplitude. A NMOS/PMOS transistor can be used as the LED driver. The external transmitter 1304 can include IR emitters for radiating the modulated IR signals at different wavelengths. Each IR emitter corresponds to a different channel in the implant TTF device.

The TTF implant 1302 harvests energy from the electromagnetic signal using a wireless element (coil or antenna). At the same time, the implant 1302 receives IR energy and converts it into a TTF signal at the corresponding channel. The TTF implant 1302 includes a wireless element for receiving wireless power. The implant 1302 can include a matching network for achieving the maximum power transfer. The implant 1302 can include a rectifier for converting the high-frequency AC signal into a DC voltage. The implant 1302 can include a power management circuit for accumulating electrical charges and providing power supply to other circuits. It can be implemented by a PMU circuit or a large capacitor. The implant 1302 can include IR detectors of different wavelengths for selectively converting incident IR signals into electrical signals. Each IR detector should only be responsive to the IR signal of specific wavelengths. Optical filters can be used to enhance channel selectivity. The implant 1302 can include modulators to generate the corresponding TTF signal from the received IR. Since the IR modulation signal is in the same frequency as the TTF signal (100-200 KHz), the modulator can also be an amplifier. It can be an MOS transistor, an Opamp, or a power amplifier. The implant 1302 can include lead electrodes for delivering the TTF field into the brain tissue. Besides IR, other wireless transmission media, such as ultrasound can also be used. The majority of the circuit can remain the same, and the transducer (IR emitter and detector) will can be replaced correspondingly.

Stimulation from the implants described herein (e.g., implants 102, 202, 302, 1202, 1302 via the electrodes) may be used to treat brain dysfunction, and the stimulation can be used for several purposes that can depend on the rate of stimulation and location of the implant (e.g., implant 102, 202, 302, 1202, 1302). Stimulation excites the underlying tissue, and thereby changes the underlying tissue's involvement in ongoing cortical processes. The effect of stimulation can depend on the electrode location, and on the stimulation rate. In some aspects, low-rate stimulation (e.g. 1 Hz) can decrease the overall excitability of a region, while higher-rate stimulation (e.g. 100 Hz) can increase the excitability of a region.

Stimulation from the implants described herein (e.g., implants 102, 202, 302, 1202, 1302 via the electrodes) can be used in several example aspects. For example, stimulation can be used in open-loop stimulation (no recording necessary), with all electrodes activated simultaneously. This stimulation may be applied at a constant rate (e.g. 1 Hz, or 100 Hz), or may be applied as “bursts” of stimulation (e.g. a burst of 3 pulses at 50 Hz, repeated a burst rate of 5 Hz).

In another example, stimulation can be used in open-loop stimulation, with multiple electrodes activated at different times. This mode could be applied as constant-rate or as burst stimulation. This mode could be used to strengthen or diminish connections between two areas and/or regions (e.g., areas and/or regions in a brain of a subject).

In another example, stimulation can be used in closed-loop stimulation (i.e., feedback stimulation), with recording that triggers stimulation when certain signals are detected. This could use a single stimulation time or stimulate multiple areas at different time. In this example, the implant (e.g., implants 102, 202, 302, 1202, 1302) can monitor one or more areas and/or regions of the brain of the subject, and, responsive to the biosignals recorded during the monitoring, the implant (e.g., implants 102, 202, 302, 1202, 1302) can apply stimulation to the monitored area or areas of the brain. The monitoring can utilize thresholds, activity monitoring, and other monitoring parameters to determine if and when to apply stimulation in response to the monitored biosignals.

In an example embodiment, the systems described herein can be used for epilepsy treatment. For example, the systems (e.g., implants 102, 202, 302, 1202, 1302 and external devices) can be used to stimulate seizure-prone cortical areas in order to disrupt pre-seizure oscillations before they can build and spread into a full seizure. In some aspects, steady stimulation, burst stimulation, or feedback stimulation in order to apply disruptive pulses to interrupt a nascent seizure.

In an example embodiment, the systems described herein can be used for rehabilitation enhancement. For example, the systems (e.g., implants 102, 202, 302, 1202, 1302 and external devices) can be used to use apply excitatory stimulation to increase the activity in an injury-weakened cortical area. This could increase the injury-weakened area's involvement in re-learning lost function during rehabilitation training, resulting in improved recovery. In some aspects, examples could use suppressive stimulation of non-injured cortical areas that could prevent the non-injured areas from suppressing the injured cortex, thereby encouraging the injured cortex to be involved in controlling activity during rehabilitation training. Some examples could use open-loop stimulation with steady stimulation to modulate cortical activity during rehabilitation. Some examples could use open-loop burst stimulation to modulate the connection between multiple cortical areas. For example, stimulating Area 1 followed by Area 2 could strengthen excitatory connections from Area 1 to Area 2. Some examples can use closed-loop stimulation to strengthen or weaken connections between cortical areas, by changing the stimulation of one area depending on the activity of another area.

Embodiments of the subject matter and the functional operations described in this specification can be implemented at least in part in digital electronic circuitry, in tangibly-embodied computer software or firmware, in computer hardware, including the structures disclosed in this specification and their structural equivalents, or in combinations of one or more of them. Embodiments of the subject matter described in this specification can be implemented at least in part as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. Computers suitable for the execution of a computer program can be based on general or special purpose microprocessors or both, or any other kind of central processing unit. Generally, a central processing unit will receive instructions and data from a read-only memory or a random access memory or both.

Computer-readable media suitable for storing computer program instructions and data include all forms of non-volatile memory, media and memory devices, including by way of example semiconductor memory devices.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A system for brain stimulation and monitoring, the system comprising:
an implant comprising:
an implant transceiver;
a power management and storage unit;
one or more electrodes;
a recorder unit; and
a memory;
an external device comprising:
a power source; and
an external device transceiver configured to send power signals and stimulation signals to the implant transceiver;
wherein the external device is configured to wirelessly send power signals and stimulation signals to the implant;
wherein the implant is configured to record one or more biosignals from the one or more electrodes and wirelessly communicate the biosignals to the external device.

2. The system of claim 1, wherein the implant is battery-less.

3. The system of claim 1 wherein the implant comprises a flexible substrate that connects to the one or more electrodes, the implant transceiver, and the power management and storage unit.

4. The system of claim 3, wherein the flexible substrate comprises a biocompatible material.

5. The system of claim 3, wherein the implant is configured to be positioned between a bone layer and a cortex of a skull of a subject.

6. A method of stimulating and monitoring a brain of a subject, the method comprising:
positioning an implant between a bone layer and a cortex of a skull of a subject;
communicating wireless power signals from an external device to the implant;
converting the wireless power signals into stored power at the implant;
communicating wireless stimulation signals from the external device to the implant;
processing the stimulation signals at the implant into stimulation voltages delivered by one or more electrodes at the implant;
recording one or more biosignals at the one or more electrodes;
storing the one or more biosignals at the implant; and
communicating the one or more biosignals wirelessly from the implant to the external device.

7. The method of claim 6, wherein the implant is battery-less.

8. The method of claim 6 or claim 7, wherein the implant comprises a flexible substrate that connects to the one or more electrodes, a transceiver, and a power management and storage unit.

9. The method of claim 8, wherein the flexible substrate comprises a biocompatible material.

10. A device for brain stimulation and monitoring, the device comprising:
an implant configured to be positioned between a bone layer and a cortex of a skull of a subject, the implant comprising:
a flexible substrate comprising a biocompatible material;
an implant transceiver connected to the flexible substrate;
a power management and storage unit connected to the flexible substrate;
one or more electrodes connected to the flexible substrate, the one or more electrodes are configured to deliver stimulation voltages to the cortex of the subject and to record biosignals from the cortex of the subject;
a recorder unit connected to the flexible substrate; and
a memory connected to the flexible substrate;
wherein the implant is configured to wirelessly communicate with an external device.

11. A device for brain treatment, the device comprising:
an implant configured to be positioned between a bone layer and a cortex of a skull of a subject, the implant comprising:
a flexible substrate comprising a biocompatible material;
an implant transceiver connected to the flexible substrate;
a power management and storage unit connected to the flexible substrate;
one or more electrodes connected to the flexible substrate, the one or more electrodes are configured to create an electrical field that delivers stimulation voltages to a treatment area in the cortex of the subject;
a recorder unit connected to the flexible substrate; and
a memory connected to the flexible substrate;
wherein the implant is configured to wirelessly communicate with an external device.

12. The device of claim 11, wherein the implant is battery-less.

13. The device of claim 11 or claim 12, wherein the implant communicates biosignals to the external device.

14. The device of claim 11, wherein the implant comprises a flexible substrate that connects to the one or more electrodes, the implant transceiver, and the power management and storage unit.

15. The device of claim 14, wherein the flexible substrate comprises a biocompatible material.

16. The device of claim 14, wherein the implant is configured to be positioned between a bone layer and a cortex of a skull of a subject.

17. A system for brain treatment, the system comprising:
an implant comprising:
an implant transceiver;
a power management and storage unit;
one or more electrodes;
a recorder unit; and
a memory;
an external device comprising:
a power source; and
an external device transceiver configured to send power signals and stimulation signals to the implant transceiver; and
wherein, responsive to the power signals and the stimulation signals, the implant generates an electrical field from the one or more electrodes in a treatment area.

18. The system of claim 17, wherein the treatment area is a tumor.

19. The system of claim 17 or claim 18, wherein the implant comprises a plurality of electrode pairs.

20. The system of claim 19, wherein the treatment area includes a plurality of tumors.
